(19) United States
(12) Patent Application Publication
(10) Pub. No.: US 2011/0306922 A1
(43) Pub. Date: Dec. 15, 2011

(54) LIGHT-EMITTING DEVICES FOR PHOTOTHERAPY

(76) Inventors: Sazzadur Rahman Khan, San Diego, CA (US); Amane Mochizuki, San Diego, CA (US); Sheng Li, Vista, CA (US); David T. Sisk, San Diego, CA (US); Rajesh Mukherjee, Irvine, CA (US); Keisaku Okada, San Diego, CA (US)

(21) Appl. No.: 13/158,055

(22) Filed: Jun. 10, 2011

Related U.S. Application Data
(60) Provisional application No. 61/353,752, filed on Jun. 11, 2010.

(51) Int. Cl. A61M 37/00; A61N 5/06; C09K 11/06

(52) U.S. Cl. 604/20; 252/301.16; 607/88

(57) ABSTRACT

Some embodiments provide a compound represented by Formula 1. Other embodiments provide an organic light-emitting diode device, such as a light-emitting device for phototherapy, comprising a compound of Formula 1. Other embodiments provide an organic light-emitting device optionally comprising a wavelength convertor. Methods related to the treatment of diseases using phototherapy area also described.

### CROSS-REFERENCE TO RELATED APPLICATIONS

This application claims the benefit of U.S. Provisional Application No. 61/353,752, filed Jun. 11, 2010, which is incorporated by reference herein in its entirety.

### BACKGROUND OF THE INVENTION

1. Field of the Invention

The embodiments relate to light-emitting devices, such as those containing organic light-emitting diodes, for uses such as phototherapy.

2. Description of the Related Art

Phototherapy may be useful in treating a number of medical conditions. However, light sources such as lasers, which may be used for phototherapy, may be expensive, difficult to transport, and not suitable for home or outpatient treatment. Therefore, there is a need for alternative sources of light for phototherapy which may be less expensive and more portable.

### SUMMARY OF THE INVENTION

Some embodiments relate to organic light-emitting devices which may be used for phototherapy. In some embodiments, the devices may comprise an organic light-emitting layer comprising a light-emitting component, such as a fluorescent or a phosphorescent compound. In some embodiments, the light-emitting layer may comprise a host compound, such as a substituted bipyridinyl compound, including a compound described herein. Some devices may also comprise wavelength convertor.

Some embodiments provide a device for use in phototherapy comprising: a light-emitting layer, wherein the light-emitting layer is disposed between the anode and the cathode. In some embodiments, the light-emitting layer comprises an electroluminescent coordination compound comprising a metal-ligand complex. The metal-ligand complex may comprise: a metal selected from platinum and iridium. The metal-ligand complex may further comprise at least 1 ligand which may be selected from the group consisting of optionally substituted acetoacetonate, optionally substituted picolinate, optionally substituted phenylpyridinato, optionally substituted triazolylpyridinato, optionally substituted benzothienylpyridinato, optionally substituted tetrazolylpyridinato, optionally substituted phenylisoquinolinato, optionally substituted tetra(1-pyrazolyl)borate, optionally substituted phenylquinolinyl, optionally substituted phenyloxazolinato, optionally substituted dibenzoquinoxalino, optionally substituted thiophenylisoquinolinato, optionally substituted 2,5-bis-(2′-fluorene)pyridine, optionally substituted phenylbenzothiazolato, optionally substituted fluorenylisoquinolinato, optionally substituted thienylpyridinato, optionally substituted phenylcarbazolylpyridinato, and optionally substituted carbazolylphenylpyridinato. Some embodiments may further comprise a wavelength convertor comprising. In some embodiments, the wavelength convertor may comprise yttrium aluminum garnet, yttria, titania or alumina. In some embodiments, the wavelength convertor may comprise yttrium aluminum garnet, yttria, titania or alumina and at least one dopant which is an atom or an ion of an element selected from the group consisting of Cr, Ce, Gd, La, Tb, Pr, Sm, and Eu. In some embodiments, the wavelength convertor may be configured to receive at least a portion of light emitted from the organic light-emitting diode in a wavelength range of about 350 nm to less than about 600 nm and convert at least a portion of the light received to light in a wavelength range of about 600 nm to about 800 nm.

Some embodiments provide a device for use in phototherapy comprising: a light-emitting layer comprising an electroluminescent coordination compound comprising a metal-ligand complex, wherein the metal-ligand complex comprises: a metal selected from platinum and iridium; and at least 1 ligand which may be selected from the group consisting of optionally substituted acetoacetonate, optionally substituted picolinate, optionally substituted phenylpyridinato, optionally substituted triazolylpyridinato, optionally substituted benzothienylpyridinato, optionally substituted tetrazolylpyridinato, optionally substituted phenylisoquinolinato, optionally substituted tetra(1-pyrazolyl)borate, optionally substituted phenylquinolinyl, optionally substituted phenyloxazolinato, optionally substituted dibenzoquinoxalino, optionally substituted thiophenylisoquinolinato, optionally substituted 2,5-bis-(2′-fluorene)pyridine, optionally substituted phenylbenzothiazolato, optionally substituted fluorenylisoquinolinato, optionally substituted thienylpyridinato, optionally substituted phenylcarbazolylpyridinato, and optionally substituted carbazolylphenylpyridinato; a wavelength convertor comprising: yttrium aluminum garnet, yttria, titania or alumina, and at least one dopant which is an atom or an ion of an element selected from the group consisting of Cr, Ce, Gd, La, Tb, Pr, Sm, and Eu; and wherein the wavelength convertor is configured to receive at least a portion of light emitted from the organic light-emitting diode in a wavelength range of about 350 nm to about 600 nm and convert at least a portion of the light received to light in a wavelength range of about 600 nm to about 800 nm.

Some embodiments relate to an organic light-emitting device for use in phototherapy comprising: a light-emitting layer comprising a host compound and an electroluminescent compound; wherein the light-emitting layer is disposed between the anode and the cathode, and wherein the host compound is represented by Formula 1:

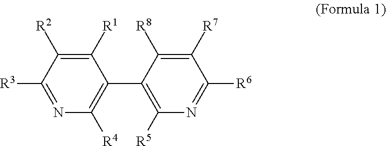

wherein R¹, R², R³, R⁶, R⁷, and R⁸ are independently selected from the group consisting of H, optionally substituted C₁₋₁₂ alkyl, optionally substituted phenyl, optionally substituted carbazolyl, optionally substituted diphenylamine, optionally substituted carbazolylphenyl, and optionally substituted diphenylaminophenyl; provided that: at least one of R¹, R², and R³ is selected from optionally substituted carbazolyl, optionally substituted diphenylamine, optionally substituted carbazolylphenyl, and optionally substituted diphenylaminophenyl, and at least one of R⁶, R⁷, and R⁸ is selected from optionally substituted carbazolyl, optionally substituted diphenylamine, optionally substituted carbazolylphenyl, and optionally substituted diphenylaminophenyl; and R⁴ and R⁵ are independently selected from the group consisting of H, optionally substituted C₁₋₁₂ alkyl, optionally substituted phenyl, optionally substituted diphenylamine and optionally substituted diphenylaminophenyl; and wherein the device is configured to emit a therapeutically effective amount of light to a mammal.

In some embodiments, these devices may be used in a method of carrying out phototherapy comprising: exposing at least a portion of a tissue of a mammal to light from a device described herein. In some embodiments, the tissue comprises a photosensitive compound which is not naturally in the tissue, and at least a portion of the photosensitive compound is activated by exposing the portion of the tissue to light from the device.

Some embodiments provide a method of treating a disease, comprising: exposing at least a portion of a tissue of a mammal in need thereof with light from a device described herein. In some embodiments, the tissue comprises a photosensitive compound which is not naturally in the tissue, and at least a portion of the photosensitive compound is activated by exposing the portion of the tissue to light from the device to thereby treat the disease.

These and other embodiments are described in greater detail below.

### BRIEF DESCRIPTION OF THE DRAWINGS

FIG. 1 is a schematic of an embodiment of a light-emitting device suitable for phototherapy which comprises a controller and a processor.

FIG. 2 shows an embodiment of a top emission light-emitting device comprising a wavelength convertor.

FIG. 3 is another schematic showing an embodiment of a top emitting light-emitting device comprising a wavelength convertor.

FIG. 4 is a schematic showing an embodiment of a bottom emitting device comprising a wavelength convertor.

FIG. 5 is a schematic showing an embodiment of a bottom emitting device in a standard manner and without wavelength convertor

FIG. 6 displays the spectroscopic properties of one embodiment of the host compound in CHCl₃ solution.

FIG. 7 is a current density vs. voltage plot of two embodiments of the bipolar host devices.

FIG. 8 is the electroluminescence spectrum of three embodiments of the light-emitting device.

FIG. 9 is a current density (mA/cm²) and/or Brightness (cd/cm2) vs. voltage (V) curve of one embodiment of the light-emitting device.

FIG. 10 shows the output power (mW/cm²) vs. applied voltage (V) of one embodiment of the light-emitting device.

FIG. 11 is a plot of the emission spectrum of an embodiment of a light-emitting device comprising a chromium doped YAG wavelength convertor and an identical light-emitting device without a wavelength convertor.

FIG. 12 is a plot of the emission spectrum of an embodiment of a light-emitting device comprising a chromium doped YAG wavelength convertor and an identical light-emitting device without a wavelength convertor.

FIG. 13 is a schematic diagram of an in vitro efficacy study with typical tumor cells.

FIG. 14 shows optical microscope images of untreated CHO-K1 cells and ALA treated CHO-K1 cells after 25 J/cm² irradiation.

FIG. 15 is a graph depicting cell viability vs. 5-ALA concentrations

FIG. 16 is a schematic diagram of the cell viability at different doses of light irradiation.

The drawings may not be to scale.

### DETAILED DESCRIPTION OF THE PREFERRED EMBODIMENT

### Definitions

As used herein, the term “alkyl” includes a hydrocarbon moiety with no double or triple bonds. Examples include, but are not limited to, linear alkyl, branched alkyl, cycloalkyl, or combinations thereof. Alkyl may be bonded to any other number of moieties (e.g. be bonded to 1 other group, such as —CH₃, 2 other groups, such as —CH₂—, or any number of other groups) that the structure may bear, and in some embodiments, may contain from one to thirty-five carbon atoms. Examples of alkyl groups include but are not limited to CH₃ (e.g. methyl), C₂H₅ (e.g. ethyl), C₃H₇ (e.g. propyl isomers such as propyl, isopropyl, etc.), C₃H₆ (e.g. cyclopropyl), C₄H₉ (e.g. butyl isomers) C₄H₈ (e.g. cyclobutyl isomers such as cyclobutyl, methylcyclopropyl, etc.), C₅H₁₁ (e.g. pentyl isomers), C₅H₁₀ (e.g. cyclopentyl isomers such as cyclopentyl, methylcyclobutyl, dimethylcyclopropyl, etc.) C₆H₁₃ (e.g. hexyl isomers), C₆H₁₂ (e.g. cyclohexyl isomers), C₇H₁₅ (e.g. heptyl isomers), C₇H₁₄ (e.g. cycloheptyl isomers), C₈H₁₇ (e.g. octyl isomers), C₈H₁₆ (e.g. cyclooctyl isomers), C₉H₁₉ (e.g. nonyl isomers), C₉H₁₈ (e.g. cyclononyl isomers), C₁₀H₂₁ (e.g. decyl isomers), C₁₀H₂₀ (e.g. cyclodecyl isomers), C₁₁H₂₃ (e.g. undecyl isomers), C₁₁H₂₂ (e.g. cycloundecyl isomers), C₁₂H₂₅ (e.g. dodecyl isomers), C₁₂H₂₄ (e.g. cyclododecyl isomers), C₁₃H₂₇ (e.g. tridecyl isomers), C₁₃H₂₆ (e.g. cyclotridecyl isomers), and the like.

Alkyl may also be defined by the following general formulas: the general formula for linear or branched alkyl containing a cyclic structure is CₙH₂ₙ₋₂, and the general formula for a fully saturated hydrocarbon containing one ring is CₙH₂ₙ. A C_{X-Y} alkyl or C_{X}-C_{y} alkyl is an alkyl having from X to Y carbon atoms. For example, C₁₋₁₂ alkyl or C₁-C₁₂ alkyl includes alkyl containing 1, 2, 3, 4, 5, 6, 7, 8, 9, 10, 11, or 12 carbon atoms.

As used herein, “optionally substituted” group refers to a group that may be substituted or unsubstituted. A substituted group is derived from the unsubstituted parent structure wherein one or more hydrogen atoms on the parent structure have been independently replaced by one or more substituent groups. A substituted group may have one or more substituent groups on the parent group structure. The substituent groups are independently selected from optionally substituted phenyl, optionally substituted alkyl, —O-alkyl (e.g. —OCH₃, —OC₂H5, —OC₃H₇, —OC₄H₉, etc.), —S-alkyl (e.g. —SCH₃, —SC₂H₅, —SC₃H₇, —SC₄H₉, etc.), —NR′R″, —OH, —SH, —CN, —NO₂, or a halogen, wherein R′ and R″ are independently H or optionally substituted alkyl. Wherever a moiety is described as “optionally substituted,” that moiety can be substituted with the above substituents.

Optionally substituted alkyl refers to unsubstituted alkyl and substituted alkyl. The substituted alkyl refers to substituted alkyl where one or more H atoms are replaced by one or more substituent groups, such as —O-alkyl (e.g. —OCH₃, —OC₂H₅, —OC₃H₇, —OC₄H₉, etc.), —S-alkyl (e.g. —SCH₃, —SC₂H5, —SC₃H₇, —SC₄H₉, etc.), —NR′R″ where R′ and R″ are independently H or alkyl, —OH, —SH, —CN, —NO₂, or a halogen. Some examples of optionally substituted alkyl may be alkyl, haloalkyl, perfluoroalkyl, hydroxyalkyl, alkylthiol (i.e. alkyl-SH), -alkyl-CN, etc.

Optionally substituted C₁₋₁₂ alkyl refers to unsubstituted C₁₋₁₂ alkyl and substituted C₁₋₁₂ alkyl. The substituted C₁₋₁₂ alkyl refers to C₁₋₁₂ alkyl where one or more hydrogen atoms are independently replaced by one or more of the substituent groups indicated above.

The term “halogen” or “halo” refers to fluoro, chloro, bromo or iodo.

The term “fluoroalkyl” refers to alkyl having one or more fluorine substituents. In other words, it is substituted alkyl where one or more hydrogen atoms are substituted by fluorine, but no other atoms except C, H, and F are present. C₁₋₆F₁₋₁₃ fluoroalkyl refers to fluoroalkyl having 1-6 carbon atoms and 1-13 fluorine atoms.

The term “perfluoroalkyl” refers to fluoroalkyl with a formula CₙF₂ₙ₊₁ for a linear or branched structure, e.g., CF₃, C₂F₅, C₃F₇, C₄F₉, C₅F₁₁, C₆F₁₃, etc., or CₙF₂ₙ for a cyclic structure, e.g., cyclic C₃F₆, cyclic C₄F₈, cyclic C₅F₁₀, cyclic C₆F₁₂, etc. In other words, every hydrogen atom in alkyl is replaced by fluorine. For example, while not intending to be limiting, C₁₋₃ perfluoroalkyl refers to CF₃, C₂F₅, and C₃F₇ isomers.

The term “optionally substituted phenyl” refers to unsubstituted phenyl or substituted phenyl. In substituted phenyl, one or more hydrogen atoms on the ring system are independently replaced by one or more substituent groups indicated above. In some embodiments, optionally substituted phenyl may be optionally substituted 1,4-interphenylene or optionally substituted 1,3-interphenylene.

The structures of some of the optionally substituted ring systems or optionally substituted ring-containing moieties referred to herein are depicted below. These ring systems or ring-containing moieties may be unsubstituted, or one or more hydrogen atoms on any ring may be independently replaced by one or more substituent groups indicated above.

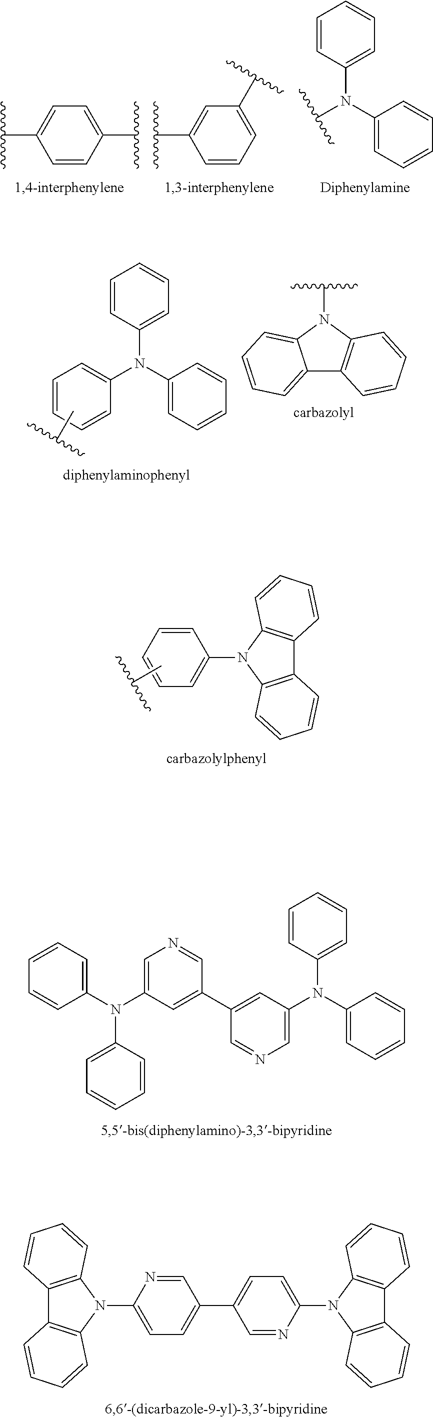

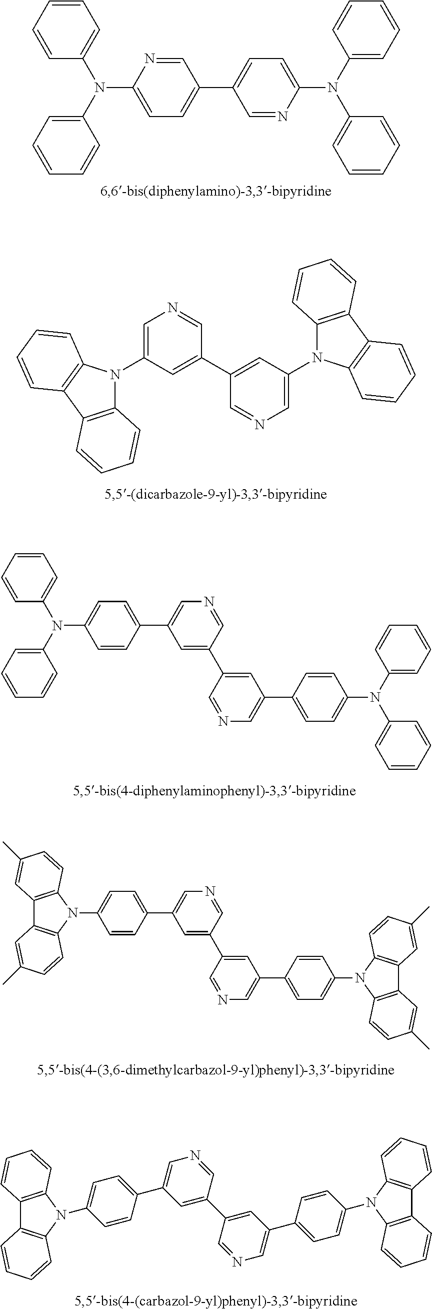

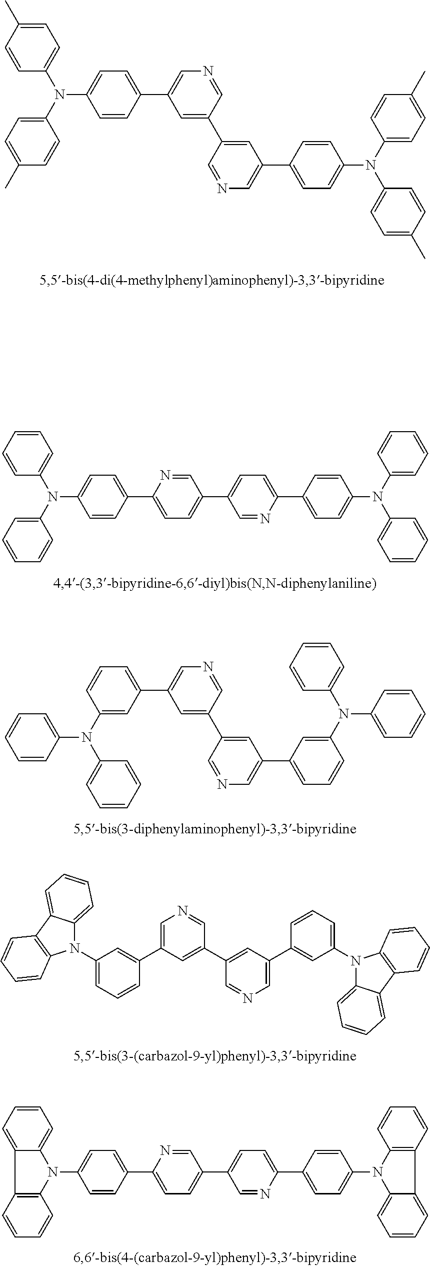

A “C2 symmetry axis” is an axis wherein rotating a molecule by 180° (i.e. 360°/2) about that axis yields the same structure. For example, in Formula 1, if: 1) R¹ is the same as R⁸, 2) R² is the same as R⁷, 3) R³ is the same as R⁶, and 4) R⁴ is the same as R⁵, then the molecule has a C2 symmetry axis.

The term “ambipolar material” refers to a material that is capable of transferring both holes and electrons effectively.

The term “phosphorescent material” refers to a material that can emit light from both singlet and triplet excitons.

The term “phototherapy” has the broadest ordinary meaning understood by a person of ordinary skill in the art, and includes any therapeutic procedure which uses light, such as using light in the diagnosis, cure, mitigation, treatment, or prevention of disease in man or other animals, or otherwise using light in a manner intended to affect the structure or any function of the body of man or other animals.

The embodiments provide a compound represented by Formula 1:

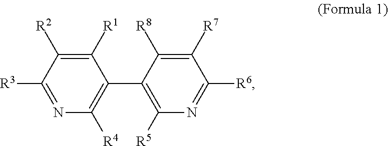

With respect to Formula 1, R¹, R², R³, R⁶, R⁷, and R⁸ are independently selected from the group consisting of H, optionally substituted C₁₋₁₂ alkyl such as optionally substituted methyl, optionally substituted ethyl, optionally substituted propyl isomers, optionally substituted cyclopropyl, optionally substituted butyl isomers, optionally substituted cyclobutyl isomers (such as cyclobutyl, methylcyclopropyl, etc.), optionally substituted pentyl isomers, optionally substituted cyclopentyl isomers, optionally substituted hexyl isomers, optionally substituted cyclohexyl isomers, optionally substituted heptyl isomers, optionally substituted cycloheptyl isomers; optionally substituted octyl isomers, optionally substituted cyclooctyl isomers, optionally substituted nonyl isomers, optionally substituted cyclononyl isomers, optionally substituted decyl isomers, optionally substituted cyclodecyl isomers, or the like; optionally substituted phenyl; optionally substituted carbazolyl; optionally substituted diphenylamine; and optionally substituted diphenylaminophenyl. In some embodiments, R¹, R², R³, R⁶, R⁷, and R⁸ are independently selected from the group consisting of H, unsubstituted C₁₋₁₂ alkyl, C₁₋₁₂ alkyl having from 1 to 13 halogen substituents (such as CF₃, C₂F₅, C₃F₇, C₄F₉, C₅F₁₁, C₆F₁₃, cyclic C₃F₆, cyclic C₄F₈, cyclic C₅F₁₀, cyclic C₆F₁₂, etc.), optionally substituted phenyl, optionally substituted carbazolyl, optionally substituted diphenylamine, optionally substituted carbazolylphenyl, and optionally substituted diphenylaminophenyl. In some embodiments, R¹, R³, R⁴, R⁵, R⁶, and R⁸ are H, and R² and R⁷ are independently selected from optionally substituted carbazolyl, optionally substituted diphenylamine, optionally substituted carbazolylphenyl, and optionally substituted diphenylaminophenyl.

With respect to Formula 1, at least one of R¹, R², and R³ is selected from optionally substituted carbazolyl, optionally substituted diphenylamine, optionally substituted carbazolylphenyl, and optionally substituted diphenylaminophenyl and at least one of R⁶, R⁷, and R⁸ is selected from optionally substituted carbazolyl, optionally substituted diphenylamine, optionally substituted carbazolylphenyl, and optionally substituted diphenylaminophenyl.

With respect to Formula 1, R⁴ and R⁵ are independently selected from the group consisting of H, optionally substituted C₁₋₁₂ alkyl, optionally substituted phenyl, optionally substituted diphenylamine and optionally substituted diphenylaminophenyl.

In Formula 1, each pyridinyl ring of the bipyridine substructure has at least one optionally substituted carbazolyl, optionally substituted diphenylamine, optionally substituted carbazolylphenyl, or optionally substituted diphenylaminophenyl which is located in a position other than the ortho position between the ring nitrogen and the carbon that connects the two rings (i.e. the position of R⁴ and R⁵). In some embodiments, R² and R⁷ are independently selected from optionally substituted carbazolyl, optionally substituted diphenylamine, optionally substituted carbazolylphenyl, or optionally substituted diphenylaminophenyl. In other embodiments, R³ and R⁶ are optionally substituted carbazolyl, optionally substituted diphenylamine, optionally substituted carbazolylphenyl, or optionally substituted diphenylaminophenyl.

In some embodiments, R² and R⁷ are independently selected from optionally substituted carbazolyl, optionally substituted diphenylamine, optionally substituted carbazolylphenyl, or optionally substituted diphenylaminophenyl, and R¹, R³, R⁶, and R⁸ are independently H, C₁₋₈ alkyl, or C₁₋₃ perfluoroalkyl. In other embodiments, R³ and R⁶ are optionally substituted carbazolyl, optionally substituted diphenylamine, optionally substituted carbazolylphenyl, or optionally substituted diphenylaminophenyl, and R¹, R², R⁷, and R⁸ are independently H, C₁₋₈ alkyl, or C₁₋₃ perfluoroalkyl. In other embodiments, R² and R⁷ are selected from optionally substituted carbazolyl, optionally substituted diphenylamine, optionally substituted carbazolylphenyl, and optionally substituted diphenylaminophenyl, and R¹, R³, R⁶, and R⁸ are H. In other embodiments, R³ and R⁶ are optionally substituted carbazole, and R¹, R², R⁷, and R⁸ are H. In some embodiments, R⁴ and R⁵ are H. In other embodiments, R¹, R³, R⁴, R⁵, R⁶, and R⁸ are H, and R² and R⁷ are optionally substituted carbazolyl.

In some embodiments, the optionally substituted C₁₋₁₂ alkyl is unsubstituted C₁₋₁₂ alkyl, or C₁₋₁₂ alkyl substituted by 1 to 13 halogen atoms.

In some embodiments, the compound of Formula 1 has a C2 symmetry axis. In other embodiments, the compound of Formula 1 does not have a C2 symmetry axis.

Some embodiments provide a compound represented by Formula 2:

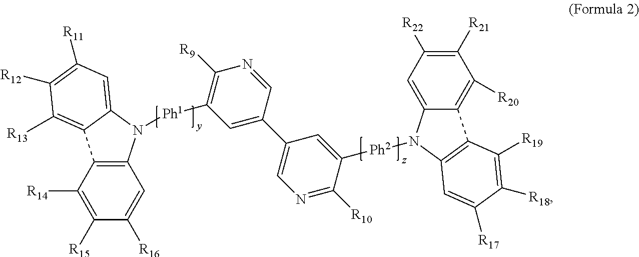

With respect to Formula 2, each dotted line is independently an optional bond. For example, some embodiments relate to compounds represented by Formula 2A or Formula 2B.

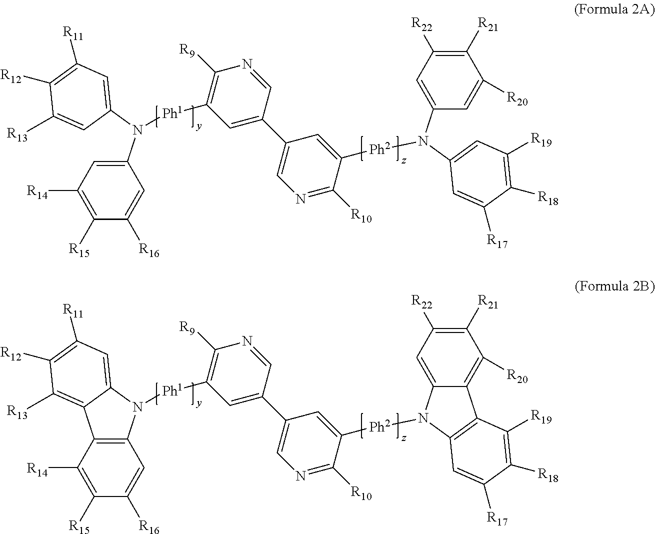

In embodiments related to Formula 2, Formula 2A, and Formula 2B, Ph¹ and Ph² are independently optionally substituted 1,4-interphenylene or optionally substituted 1,3-interphenylene. In some embodiments, Ph¹ and Ph² may have 1, 2, or 3 substituents independently selected from C₁₋₆ alkyl and C₁₋₆ perfluoroalkyl.

Furthermore, with respect to Formula 2, Formula 2A, and Formula 2B, y may be 0 or 1 and z may be 0 or 1. R⁹ and R¹⁰ are independently H, C₁₋₃ alkyl, or C₁₋₃ perfluoroalkyl; and R¹¹, R¹², R¹³, R¹⁴, R¹⁵, R¹⁶, R¹⁷, R¹⁸, R¹⁹, R²⁰, R²¹, and R²² are independently selected from the group consisting of H, C₁₋₁₂ alkyl, C₁₋₆F₁₋₁₃ fluoroalkyl, and optionally substituted phenyl.

With respect to Formula 2, Formula 2A, and Formula 2B, in some embodiments, R⁹ and R¹⁰ are H; R⁹ and R¹⁰ are CH₃; or, alternatively, R⁹ and R¹⁰ are CF₃. In other embodiments, R¹¹ is C₁₋₈ alkyl, or alternatively, phenyl. In other embodiments, R¹² is C₁₋₈ alkyl, or alternatively, phenyl. In other embodiments, R¹¹, R¹⁶, R¹⁷, and R²² are independently H or C₁₋₈ alkyl. In some embodiments, R¹¹, R¹⁶, R¹⁷, and R²² are independently C₁₋₈ alkyl or phenyl. In some embodiments, R¹¹, R¹⁶, R¹⁸, and R²¹ are independently H, C₁₋₈ alkyl or phenyl. In some embodiments, R¹², R¹⁵, R¹⁸, and R²¹ are independently H, C₁₋₈ alkyl or phenyl.

Other embodiments provide a compound selected from optionally substituted 5,5′-bis(diphenylamino)-3,3′-bipyridine, optionally substituted 6,6′-(dicarbazole-9-yl)-3,3′-bipyridine, optionally substituted 6,6′-bis(diphenylamino)-3,3′-bipyridine, optionally substituted 5,5′-(dicarbazole-9-yl)-3,3′-bipyridine, optionally substituted 5,5′-bis(4-diphenylaminophenyl)-3,3′-bipyridine, optionally substituted 5,5′-bis(4-(3,6-dimethylcarbazol-9-yl)phenyl)-3,3′-bipyridine, optionally substituted 5,5′-bis(4-(carbazol-9-yl)phenyl)-3,3′-bipyridine, optionally substituted 5,5′-bis(4-di(4-methylphenyl)aminophenyl)-3,3′-bipyridine, optionally substituted 4,4′-(3,3′-bipyridine-6,6′-diyl)bis(N,N-diphenylaniline), optionally substituted 5,5′-bis(3-diphenylaminophenyl)-3,3′-bipyridine, optionally substituted 5,5′-bis(3-(carbazol-9-yl)phenyl)-3,3′-bipyridine, and optionally substituted 6,6′-bis(4-(carbazol-9-yl)phenyl)-3,3′-bipyridine. In some embodiments, these compounds may be unsubstituted, or have 1, 2, 3, 4, 5, or 6 substituents independently selected from: C₁₋₁₂ alkyl; CF₃; and phenyl having 0, 1, or 2 substituents, wherein the substituents on phenyl are independently C₁₋₃ alkyl or CF₃.

Some embodiments provide a compound represented by Formula 3:

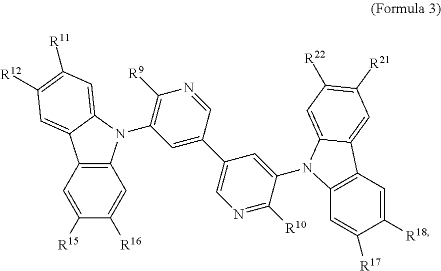

wherein R⁹ and R¹⁰ are independently H, CH₃, or CF₃; and R¹¹, R¹², R¹⁵, R¹⁶, R¹⁷, R¹⁸, R²¹, and R²² are independently H, unsubstituted phenyl, or C₁₋₈ alkyl. In some embodiments, R¹¹, R¹⁶, R¹⁷, and R²² are independently H, C₁₋₈ alkyl, or phenyl. In other embodiments, R¹¹, R¹⁶, R¹⁸, and R²¹ are independently H, C₁₋₈ alkyl or phenyl. In other embodiments, R¹², R¹⁵, R¹⁸, and R²¹ are independently H, C₁₋₈ alkyl or phenyl.

Some embodiments provide one of the compounds below.

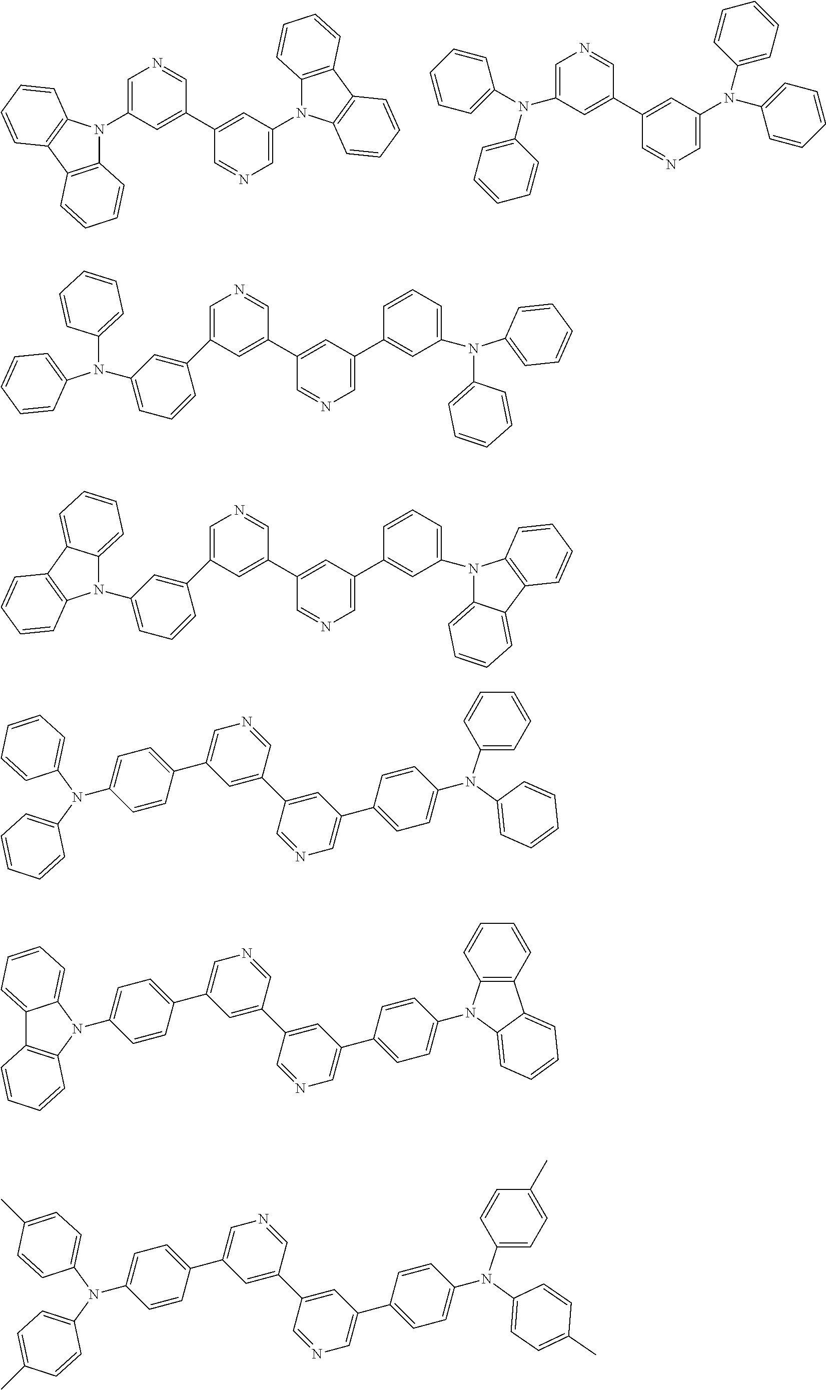

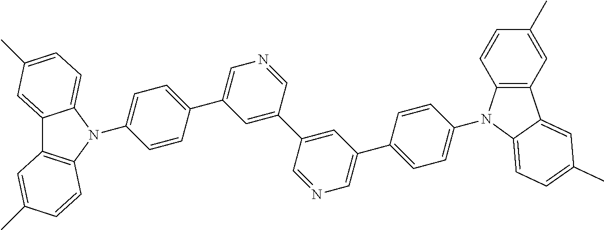

The compounds described herein can be incorporated into light-emitting devices in various ways. A light-emitting device may have a cathode, an anode, and an organic component comprising a compound described herein. At least one of the compounds described herein may be present in the organic component, and may be useful as a host material with electron-transfer properties, hole-transfer properties, or both electron-transfer and hole-transfer properties.

In some embodiments, the organic component comprises a light-emitting layer, and the device may be configured to allow holes to be transported from the anode to the light-emitting layer and allow electrons to be transported from the cathode to the light-emitting layer. The light-emitting layer may optionally comprise the host compound. Additionally, the organic component may further comprise a hole-transport layer disposed between the anode and the light-emitting layer, and which may be configured to allow holes to be transported from the anode to the light-emitting layer. The organic component may further comprise an electron-transport layer disposed between the cathode and the light-emitting layer, which may be configured to allow electrons to be transported from the cathode to the light-emitting layer.

In some embodiments, at least one of the light-emitting layer, the hole-transport layer and the electron-transport layer comprise the host compound. In some embodiments, all of the light-emitting layer, the hole-transport layer and the electron-transport layer comprise the host compound. In one embodiment, the host is ambipolar, and its ability to transfer holes is about equal to its ability to transport electrons.

An anode layer may comprise a conventional material such as a metal, mixed metal, alloy, metal oxide or mixed-metal oxide, or a conductive polymer. Examples of suitable metals include the metals in Groups 10, Group 11, and Group 12 transition metals. If the anode layer is to be light-transmitting, mixed-metal oxides of Groups 12, Group 13, and Group 14 metals or alloys thereof, such as zinc oxide, tin oxide, indium zinc oxide (IZO) or indium-tin-oxide (ITO) may be used. The anode layer may include an organic material such as polyaniline, e.g., as described in “Flexible light-emitting diodes made from soluble conducting polymer,” Nature, vol. 357, pp. 477-479 (11 Jun. 1992). Examples of suitable high work function metals include but are not limited to Au, Pt, indium-tin-oxide (ITO), or alloys thereof. In some embodiments, the anode layer can have a thickness in the range of about 1 nm to about 1000 nm.

A cathode layer may include a material having a lower work function than the anode layer. Examples of suitable materials for the cathode layer include those selected from alkali metals of Group 1, Group 2 metals, Group 11, Group 12, and Group 13 metals including rare earth elements, lanthanides and actinides, materials such as aluminum, indium, calcium, barium, samarium and magnesium, and combinations thereof. Li-containing organometallic compounds, LiF, and Li2O may also be deposited between the organic layer and the cathode layer to lower the operating voltage. Suitable low work function metals include but are not limited to Al, Ag, Mg, Ca, Cu, Mg/Ag, LiF/Al, CsF, CsF/Al or alloys thereof. In some embodiments, the cathode layer can have a thickness in the range of about 1 nm to about 1000 nm.

In some embodiments, the light-emitting layer may further comprise a light-emitting component or compound. The light-emitting component may be a fluorescent and/or a phosphorescent compound. In some embodiments, the light-emitting component comprises a phosphorescent material.

In some embodiments, the light-emitting component or compound may comprise an electroluminescent coordination compound comprising a metal-ligand complex. The metal ligand complex may comprise a metal such as platinum, iridium, osmium, ruthenium, europium etc. The metal ligand complex may also comprise 1, 2, 3, 4, 5, 6, 7, 8, 9, 10, 11, 12, or more ligands. Some non-limiting examples of ligands may include at least one of optionally substituted acetoacetonate, optionally substituted picolinate, optionally substituted phenylpyridinato, optionally substituted triazolylpyridinato, optionally substituted benzothienylpyridinato, optionally substituted tetrazolylpyridinato, optionally substituted phenylisoquinolinato, optionally substituted tetra(1-pyrazolyl)borate, optionally substituted phenylquinolinyl, optionally substituted phenyloxazolinato, optionally substituted dibenzoquinoxalino, optionally substituted thiophenylisoquinolinato, optionally substituted 2,5-bis-(2′-fluorene)pyridine, optionally substituted phenylbenzothiazolato, optionally substituted fluorenylisoquinolinato, optionally substituted thienylpyridinato, optionally substituted phenylcarbazolylpyridinato, optionally substituted carbazolylphenylpyridinato, etc.

Example structures of some of the optionally substituted ligands referred to herein are depicted below. The names are broader than the structures, and may thus include structures, such as isomers, not depicted below. These ligands may be unsubstituted, or one or more hydrogen atoms on the ligand may be independently replaced by one or more substituent groups indicated above.

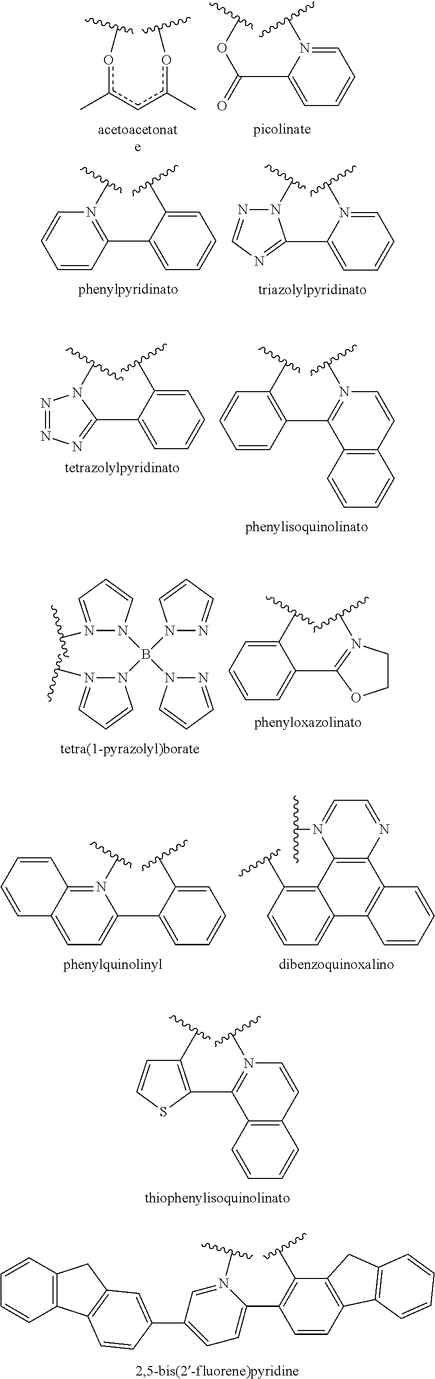

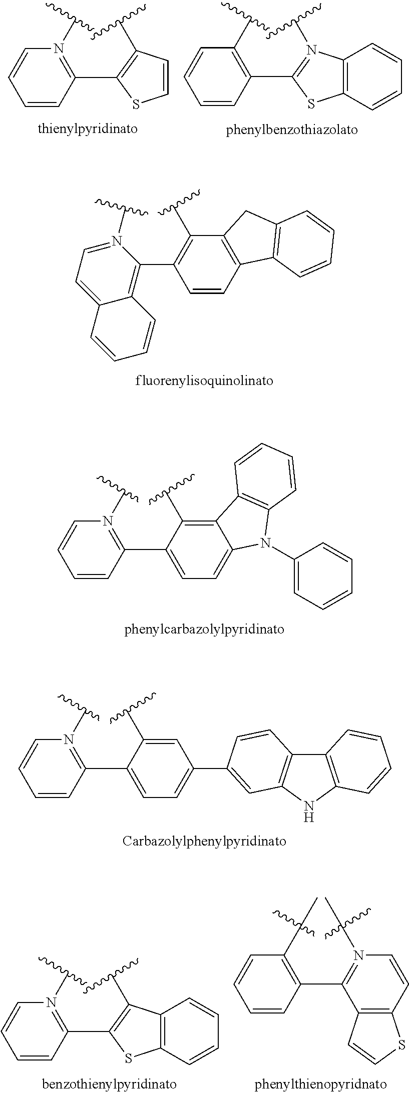

The light-emitting component or compound may be chosen to vary the color of the light emitted by the light-emitting device. For example, a blue light-emitting component may emit a combination of visible photons so that the light appears to have a blue quality to an observer. In some embodiments, a blue light-emitting component may emit visible photons having an average wavelength in the range of about 440 nm or about 460 nm to about 490 nm or about 500 nm. Some non-limiting examples of compounds which may form part or all of a blue light-emitting component include iridium coordination compounds such as: bis-{2-[3,5-bis(trifluoromethyl)phenyl]pyridinato-N, C2′}iridium(III)-picolinate, bis(2-[4,6-difluorophenyl]pyridinato-N,C2′)iridium (III) picolinate, bis(2-[4,6-difluorophenyl]pyridinato-N,C2′)iridium(acetylacetonate), Iridium (III) bis(4,6-difluorophenylpyridinato)-3-(trifluoromethyl)-5-(pyridine-2-yl)-1,2,4-triazolate, Iridium (III) bis(4,6-difluorophenylpyridinato)-5-(pyridine-2-yl)-1H-tetrazolate, bis[2-(4,6-difluorophenyl)pyridinato-N,C²]iridium(III)tetra(1-pyrazolyl)borate, etc.

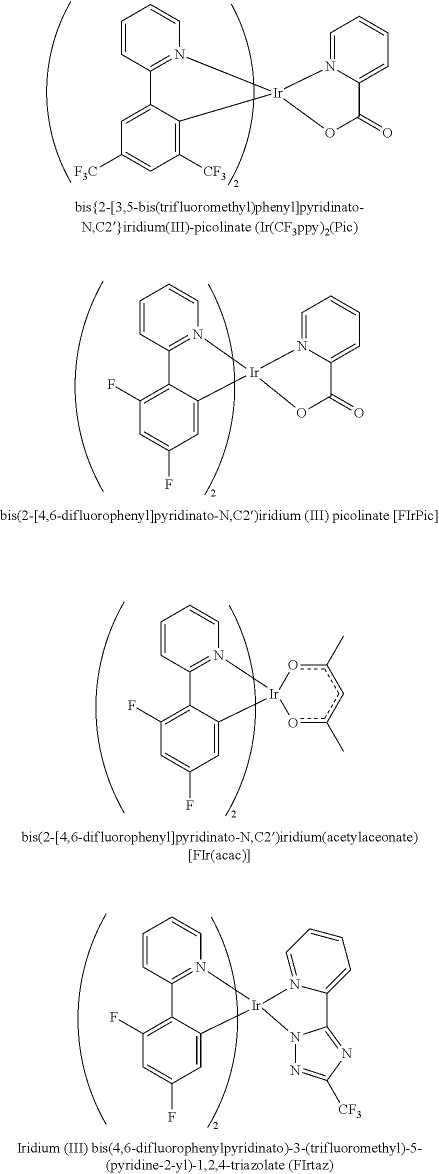

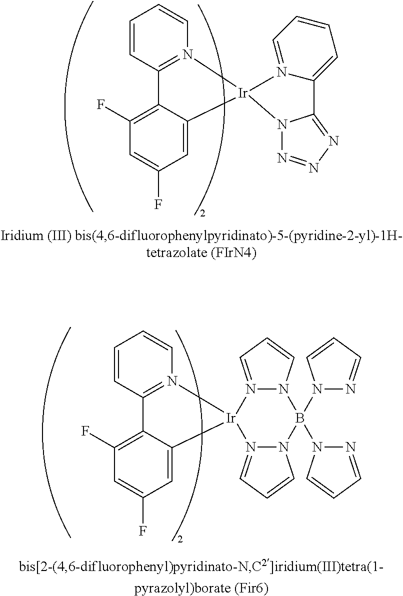

A red light-emitting component may emit a combination of visible photons so that the light appears to have a red quality to an observer. In some embodiments, a red light-emitting component may emit visible photons having an average wavelength in the range of about 600 nm or about 620 nm to about 780 nm or about 800 nm. Some non-limiting examples of compounds which may form part or all of a red light-emitting component include iridium coordination compounds such as: Bis[2-(2′-benzothienyl)-pyridinato-N,C3′]iridium (III)(acetylacetonate); Bis[(2-phenylquinolyl)-N, C2′]iridium (III) (acetylacetonate); Bis[(1-phenylisoquinolinato-N, C2′)]iridium (III) (acetylacetonate); Bis[(dibenzo[f,h]quinoxalino-N, C2′) iridium (III)(acetylacetonate); Tris(2,5-bis-2′-(9′,9′-dihexylfluorene)pyridine)iridium (III); Tris[1-phenylisoquinolinato-N, C2′]iridium (III); Tris-[2-(2′-benzothienyl)-pyridinato-N, C3′]iridium (III); Tris[1-thiophen-2-ylisoquinolinato-N, C3′]iridium (III); and Tris[1-(9,9-dimethyl-9H-fluoren-2-yl)isoquinolinato-(N,C3′)iridium (III)), etc.

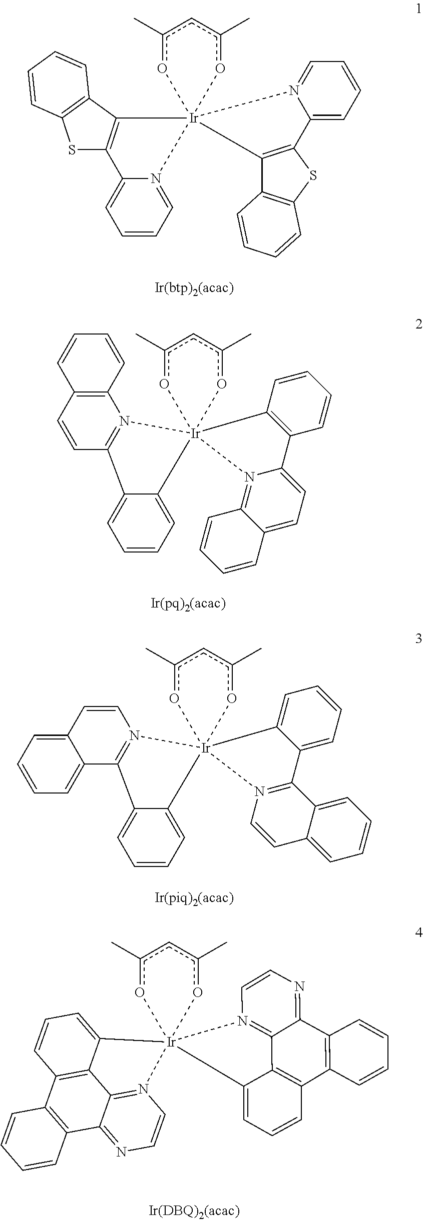

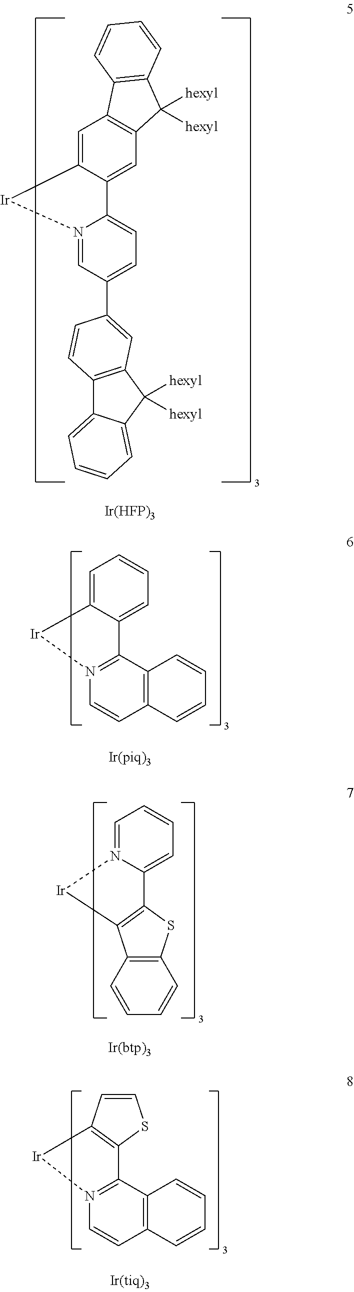

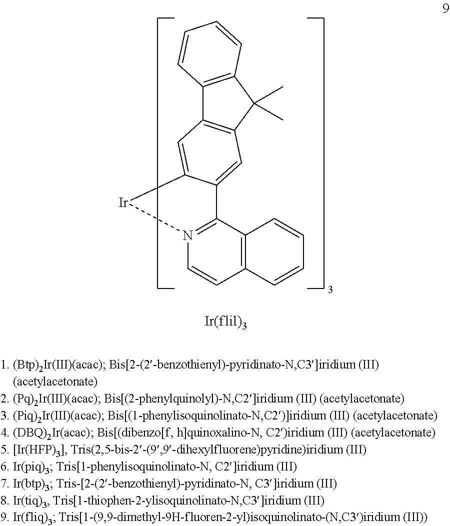

A green light-emitting component may emit a combination of visible photons so that the light appears to have a green quality to an observer. In some embodiments, a green light-emitting component may emit visible photons having an average wavelength in the range of about 490 nm or about 500 nm to about 570 nm or about 600 nm. Some non-limiting examples of compounds which may form part or all of a green light-emitting component include iridium coordination compounds such as: Bis(2-phenylpyridinato-N, C2′) iridium(III)(acetylacetonate) [Ir(ppy)₂(acac)], Bis(2-(4-tolyl)pyridinato-N, C2′) iridium(III)(acetylacetonate) [Ir(mppy)₂(acac)], Bis(2-(4-tert-butyl)pyridinato-N,C2′)iridium (III)(acetylacetonate) [Ir(t-Buppy)₂(acac)], Tris(2-phenylpyridinato-N,C2′)iridium (III) [Ir(ppy)₃], Bis(2-phenyloxazolinato-N,C2′)iridium (III) (acetylacetonate) [Ir(op)₂(acac)], Tris(2-(4-tolyl)pyridinato-N,C2′)iridium(III) [Ir(mppy)₃], etc.

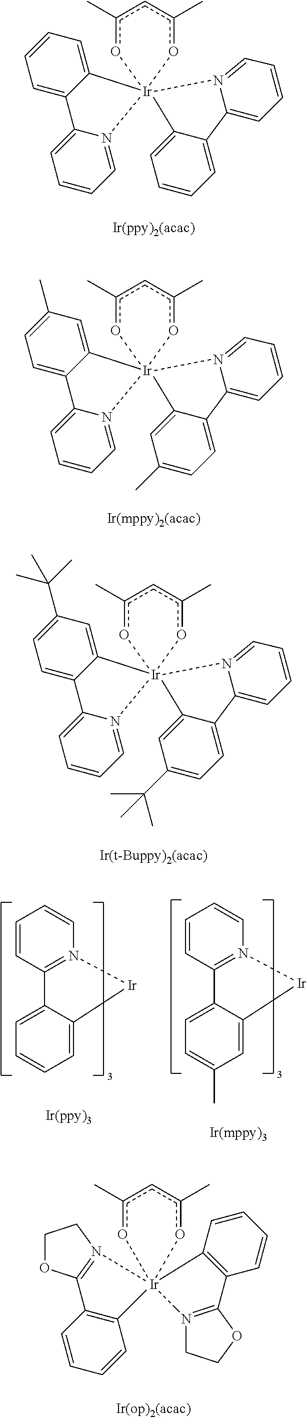

An orange light-emitting component may emit a combination of visible photons so that the light appears to have a orange quality to an observer. In some embodiments, an orange light-emitting component may emit visible photons having an average wavelength in the range of about 570 nm or about 585 nm to about 620 nm or about 650 nm. Some non-limiting examples of compounds which may form part or all of an orange light-emitting component include iridium coordination compounds such as: Bis[2-phenylbenzothiazolato-N, C2′]iridium (III)(acetylacetonate), Bis[2-(4-tert-butylphenyl)benzothiazolato-N, C2′]iridium(III)(acetylacetonate), Bis[(2-(2′-thienyl)pyridinato-N, C3′)]iridium (III) (acetylacetonate), Tris[2-(9,9-dimethylfluoren-2-yl)pyridinato-(N, C3′)]iridium (III), Tris[2-(9,9-dimethylfluoren-2-yl)pyridinato-(N, C3′)]iridium (III), Bis[5-trifluoromethyl-2-[3-(N-phenylcarbazolyl)pyridinato-N, C2′]iridium(III)(acetylacetonate), (2-PhPyCz)₂Ir(III)(acac), Bis[4-phenylthieno[3,2-c]pyridine]IrIII acetylacetonate, Ir(pthpy)₂(acac), etc.

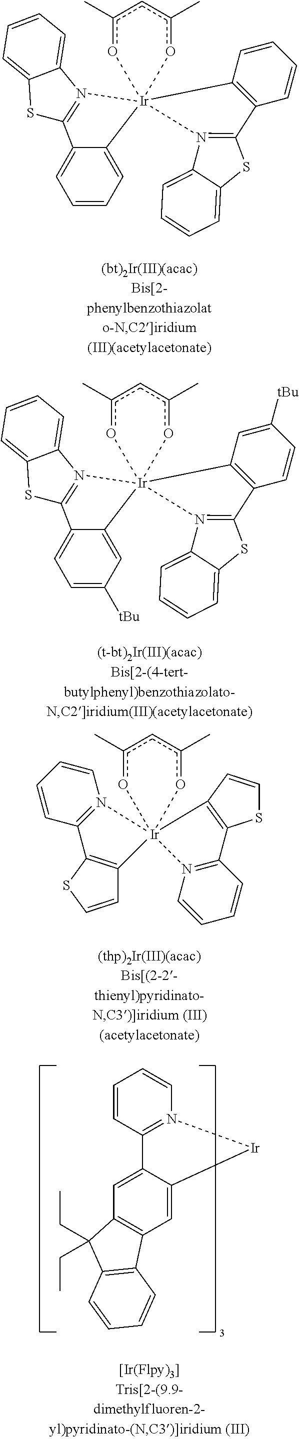

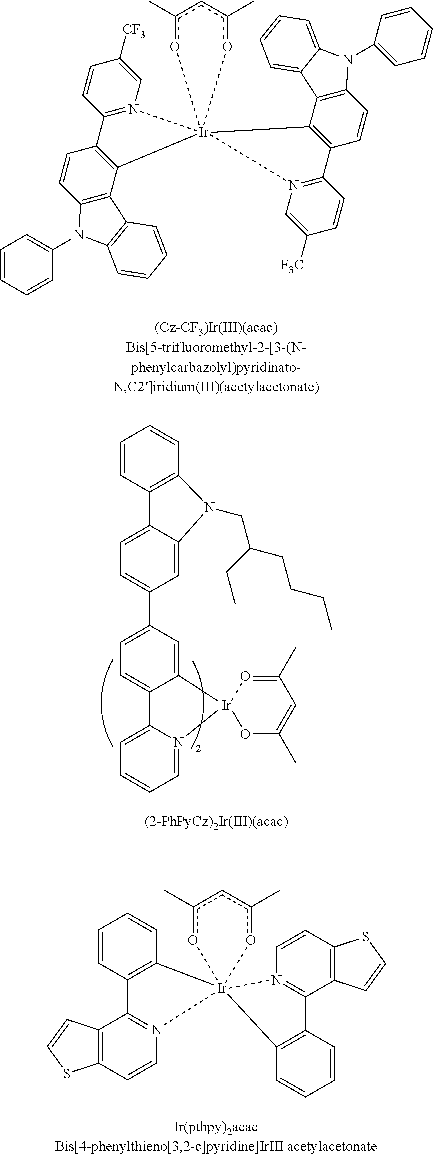

The amount of the light-emitting component may vary. In some embodiments, the light-emitting component may be about 0.1% (w/w) to about 15% (w/w), or about 9% (w/w) with respect to the host.

The thickness of the light-emitting layer may vary. In some embodiments, the light-emitting layer has a thickness from about 1 nm to about 200 nm. In some embodiments, the light-emitting layer has a thickness in the range of about 1 nm to about 100 nm.

In some embodiments, the light-emitting layer can further include additional host material. Host materials can be bipolar, for example exhibit both hole transporting and electron transporting characteristics. The host materials can also be hole dominating or electron dominating. The term “hole dominating” includes materials wherein the hole mobility within the material is greater than the electron mobility within the material. For example, the hole mobility within the material may be greater than the electron mobility within the material by at least about 10 cm²/Vs or about 100 cm²/Vs. The term “electron dominating” includes materials wherein the electron mobility within the material is greater than the hole mobility within the material. For example, the electron mobility within the material may be greater than the hole mobility within the material by at least about 10 cm²/Vs or about 100 cm²/Vs. Exemplary host materials are known to those skilled in the art. For example, the host material included in the light-emitting layer can be an optionally substituted compound selected from: an aromatic-substituted amine, an aromatic-substituted phosphine, a thiophene, an oxadiazole, 2-(4-biphenylyl)-5-(4-tert-butylphenyl)-1,3,4-oxadiazole (PBD), 1,3-bis(N,N-t-butyl-phenyl)-1,3,4-oxadiazole (OXD-7), a triazole, 3-phenyl-4-(1′-naphthyl)-5-phenyl-1,2,4-triazole (TAZ), 3,4,5-Triphenyl-1,2,3-triazole, 3,5-Bis(4-tert-butyl-phenyl)-4-phenyl[1,2,4]triazole, an aromatic phenanthroline, 2,9-dimethyl-4,7-diphenyl-phenanthroline (bathocuproine or BCP), 2,9-Dimethyl-4,7-diphenyl-1,10-phenanthroline, a benzoxazole, a benzothiazole, a quinoline, aluminum tris(8-hydroxyquinolate) (Alq3), a pyridine, a dicyanoimidazole, cyano-substituted aromatic, 1,3,5-tris(2-N-phenylbenzimidazolyl)benzene (TPBI), 4,4′-bis[N-(naphthyl)-N-phenyl-amino]biphenyl (α-NPD), N,N′-bis(3-methylphenyl)N,N′-diphenyl-[1,1′-biphenyl]-4,4′-diamine (TPD), 4,4′-bis[N,N′-(3-tolyl)amino]-3,3′-dimethylbiphenyl (M14), 4,4′-bis[N,N′-(3-tolyl)amino]-3,3′-dimethylbiphenyl (HMTPD), 1,1-Bis(4-bis(4-methylphenyl)aminophenyl)cyclohexane, a carbazole, 4,4′-N,N′-dicarbazole-biphenyl (CBP), poly(9-vinylcarbazole) (PVK), N,N′N″-1,3,5-tricarbazoloylbenzene (tCP), a polythiophene, a benzidine, N,N′-bis(4-butylphenyl)-N,N′-bis(phenyl)benzidine, a triphenylamine, 4,4′,4″-Tris(N-(naphthylen-2-yl)-N-phenylamino)triphenylamine, 4,4′,4″-tris(3-methylphenylphenylamino)triphenylamine (MTDATA), a phenylenediamine, a polyacetylene, and a phthalocyanine metal complex.

In some embodiments, the light-emitting device may further comprise a hole-transport layer between the anode and the light-emitting layer and an electron-transport layer between the cathode and the light-emitting layer. In some embodiments, all of the light-emitting layer, the hole-transport layer and the electron-transport layer comprise the host compound described herein.

In some embodiments, the hole-transport layer may comprise at least one hole-transport material. Suitable hole-transport materials are known to those skilled in the art. Exemplary hole-transport materials include: 1,1-Bis(4-bis(4-methylphenyl)aminophenyl)cyclohexane; 2,9-Dimethyl-4,7-diphenyl-1,10-phenanthroline; 3,5-Bis(4-tert-butyl-phenyl)-4-phenyl[1,2,4]triazole; 3,4,5-Triphenyl-1,2,3-triazole; 4,4′,4″-Tris(N-(naphthylen-2-yl)-N-phenylamino)triphenylamine; 4,4′,4′-tris(3-methylphenylphenylamino)triphenylamine (MTDATA); 4,4′-bis[N-(naphthyl)-N-phenyl-amino]biphenyl (α-NPD); 4,4′-bis[N,N′-(3-tolyl)amino]-3,3′-dimethylbiphenyl (HMTPD); 4,4′-bis[N,N′-(3-tolyl)amino]-3,3′-dimethylbiphenyl (M14); 4,4′-N,N′-dicarbazole-biphenyl (CBP); 1,3-N,N-dicarbazole-benzene (mCP); poly(9-vinylcarbazole) (PVK); a benzidine; a carbazole; a phenylenediamine; a phthalocyanine metal complex; a polyacetylene; a polythiophene; a triphenylamine; an oxadiazole; copper phthalocyanine; N,N′-bis(3-methylphenyl)N,N′-diphenyl-[1,1′-biphenyl]-4,4′-diamine (TPD); N,N′N″-1,3,5-tricarbazoloylbenzene (tCP); N,N′-bis(4-butylphenyl)-N,N′-bis(phenyl)benzidine; and the like.

In some embodiments, the electron-transport layer may comprise at least one electron-transport material. Suitable electron transport materials are known to those skilled in the art. Exemplary electron transport materials that can be included in the electron transport layer are an optionally substituted compound selected from: aluminum tris(8-hydroxyquinolate) (Alq3), 2-(4-biphenylyl)-5-(4-tert-butylphenyl)-1,3,4-oxadiazole (PBD), 1,3-bis(N,N-t-butyl-phenyl)-1,3,4-oxadiazole (OXD-7), 1,3-bis[2-(2,2′-bipyridine-6-yl)-1,3,4-oxadiazo-5-yl]benzene (BPY-OXD), 3-phenyl-4-(1′-naphthyl)-5-phenyl-1,2,4-triazole (TAZ),2,9-dimethyl-4,7-diphenyl-phenanthroline (bathocuproine or BCP), and 1,3,5-tris[2-N-phenylbenzimidazol-z-yl]benzene (TPBI). In one embodiment, the electron transport layer is 2-(4-biphenylyl)-5-(4-tert-butylphenyl)-1,3,4-oxadiazole (PBD), phenanthroline, quinoxaline, or a derivative or a combination thereof.

If desired, additional materials may be included in the light-emitting device. Additional materials that may be included include an electron injection materials, hole blocking materials, exciton blocking materials, and/or hole injection materials. The electron injection materials, hole blocking materials, exciton blocking materials, and/or hole injection materials may be incorporated into any of the layers described above, or may be incorporated into one or more separate layers, such as an electron injection layer, a hole blocking layer, an exciton blocking layer, and/or a hole injection layer.

In some embodiments, the light-emitting device can include an electron injection layer between the cathode layer and the light emitting layer. In some embodiments, the lowest unoccupied molecular orbital (LUMO) energy level of the electron injection material(s) is high enough to prevent it from receiving an electron from the light emitting layer. In other embodiments, the energy difference between the LUMO of the electron injection material(s) and the work function of the cathode layer is small enough to allow efficient electron injection from the cathode. A number of suitable electron injection materials are known to those skilled in the art. Examples of suitable electron injection material(s) include but are not limited to, an optionally substituted compound selected from the following: LiF, CsF, Cs doped into electron transport material as described above or a derivative or a combination thereof.

In some embodiments, the device can include a hole blocking layer, e.g., between the cathode and the light-emitting layer. Various suitable hole blocking materials that can be included in the hole blocking layer are known to those skilled in the art. Suitable hole blocking material(s) include but are not limited to, an optionally substituted compound selected from the following: 1,3,5-tris(N-phenylbenzimidizol-2-yl)benzene (TPBI), Bis(2-methyl-8-quinolinolate)-4-(phenylphenolato)aluminium (BAlq), 4,7-diphenyl-1,10-phenanthroline (BPhen), 3,4,5-triphenyl-1,2,4-triazole, 3,5-bis(4-tert-butyl-phenyl)-4-phenyl-[1,2,4]triazole, 2,9-dimethyl-4,7-diphenyl-1,10-phenanthroline (bathocuproine, BCP), and 1,1-bis(4-bis(4-methylphenyl)aminophenyl)-cyclohexane.

In some embodiments, the light-emitting device can include an exciton blocking layer, e.g., between the light-emitting layer and the anode. In one embodiment, the band gap of the exciton blocking material(s) is large enough to substantially prevent the diffusion of excitons. A number of suitable exciton blocking materials that can be included in the exciton blocking layer are known to those skilled in the art. Examples of exciton blocking material(s) include an optionally substituted compound selected from the following: aluminum quinolate (Alq₃), 4,4′-bis[N-(naphthyl)-N-phenyl-amino]biphenyl (α-NPD), 4,4′-N,N′-dicarbazole-biphenyl (CBP), and bathocuproine (BCP), and any other material(s) that have a large enough band gap to substantially prevent the diffusion of excitons.

In some embodiments, the light-emitting device can include a hole injection layer, e.g., between the light-emitting layer and the anode. Various suitable hole injection materials that can be included in the hole injection layer are known to those skilled in the art. Exemplary hole injection material(s) include an optionally substituted compound selected from the following: a polythiophene derivative such as poly(3,4-ethylenedioxythiophene (PEDOT)/polystyrene sulphonic acid (PSS), a benzidine derivative such as N,N,N′,N′-tetraphenylbenzidine, poly(N,N′-bis(4-butylphenyl)-N,N′-bis(phenyl)benzidine), a triphenylamine or phenylenediamine derivative such as N,N′-bis(4-methylphenyl)-N,N′-bis(phenyl)-1,4-phenylenediamine, 4,4′,4″-tris(N-(naphthylen-2-yl)-N-phenylamino)triphenylamine, an oxadiazole derivative such as 1,3-bis(5-(4-diphenylamino)phenyl-1,3,4-oxadiazol-2-yl)benzene, a polyacetylene derivative such as poly(1,2-bis-benzylthio-acetylene), and a phthalocyanine metal complex derivative such as phthalocyanine copper. Hole-injection materials, while still being able to transport holes, may have a hole mobility substantially less than the hole mobility of conventional hole transport materials.

Those skilled in the art would recognize that the various materials described above can be incorporated in several different layers depending on the configuration of the device. In one embodiment, the materials used in each layer are selected to result in the recombination of the holes and electrons in the light-emitting layer.

Light-emitting devices comprising the compounds disclosed herein can be fabricated using techniques known in the art, as informed by the guidance provided herein. For example, a glass substrate can be coated with a high work functioning metal such as ITO which can act as an anode. After patterning the anode layer, a light-emitting layer that includes at least a compound disclosed herein can be deposited on the anode. The cathode layer, comprising a low work functioning metal (e.g., Mg:Ag), can then be vapor evaporated onto the light-emitting layer. If desired, the device can also include an electron transport/injection layer, a hole blocking layer, a hole injection layer, an exciton blocking layer and/or a second light-emitting layer that can be added to the device using techniques known in the art, as informed by the guidance provided herein.

### Phototherapy

The devices disclosed herein may be useful in phototherapy. Typically, phototherapy involves exposing at least a portion of the tissue of a mammal with light, such as light from a device described herein.

The phototherapy may have a therapeutic effect, such as the diagnosis, cure, mitigation, treatment, or prevention of disease, or otherwise affecting the structure or function of the body of man or other animals. Some examples of conditions that phototherapy may be useful to treat or diagnose include, but are not limited to, infection, cancer/tumors, cardiovascular conditions, dermatological conditions, a condition affecting the eye, obesity, pain or inflammation, conditions related to immune response, etc.

Examples of infections may include microbial infection such as bacterial infection, viral infection, fungus infection, protozoa infection, etc.

Exemplary cancer or tumor tissues include vascular endothelial tissue, an abnormal vascular wall of a tumor, a solid tumor, a tumor of a head, a tumor of the brain, a tumor of a neck, a tumor of a gastrointestinal tract, a tumor of a liver, a tumor of a breast, a tumor of a prostate, a tumor of a lung, a nonsolid tumor, malignant cells of one of a hematopoietic tissue and a lymphoid tissue, lesions in a vascular system, a diseased bone marrow, diseased cells in which the disease is one of an autoimmune and an inflammatory disease, etc.

Examples of cardiovascular conditions may include myocardial infarction, stroke, lesions in a vascular system, such as atherosclerotic lesions, arteriovenous malformations, aneurysms, venous lesions, etc. For example, a target vascular tissue may be destroyed by cutting off circulation to the desired location.

Examples of dermatological conditions may include hair loss, hair growth, acne, psoriasis, wrinkles, discoloration, skin cancer, rosacea, etc.

Examples of eye conditions may include age related macular degeneration (AMD), glaucoma, diabetic retinopathy, neovascular disease, pathological myopia, ocular histoplasmosis, etc.

Examples of pain or inflammation include arthritis, carpal tunnel, metatarsalgia, plantar fasciitis, TMJ, pain or inflammation affecting an elbow, an ankle, a hip, a hand, etc. Examples of conditions related to immune response include, HIV or other autoimmune disease, organ transplant rejection, etc.

Other non-limiting uses of phototherapy may include treating benign prostate hyperplasia, treating conditions affecting adipose tissue, wound healing, inhibiting cell growth, and preserving donated blood.

The light itself may be at least partially responsible for the therapeutic effects of the phototherapy, thus phototherapy may be carried out without a photosensitive compound. In embodiments where a photosensitive compound is not used, light in the red range (approximately 630 nm to approximately 700 nm) may decrease inflammation in injured tissue, increase ATP production, and otherwise stimulate beneficial cellular activity. Light in the red range may also be used in conjunction with light of other spectral wavelengths, for example blue or yellow, to facilitate post operative healing. Facial rejuvenation may be effected by applying about 630 nm to about 700 nm, about 630 to about 650 nm, or about 633 nm radiation to the desired tissue for about 20 minutes. In some embodiments, facial skin rejuvenation is believed to be attained by applying about red light for a therapeutically effective amount of time.

The light may also be used in conjunction with a photosensitive compound. The photosensitive compound may be administered directly or indirectly to body tissue so that the photosensitive compound is in or on the tissue. At least a portion of the photosensitive compound may then be activated by exposing at least a portion of tissue with light.

For example, a photosensitive compound may be administered systemically by ingestion or injection, topically applying the compound to a specific treatment site on a patient's body, or by some other method. This may be followed by illumination of the treatment site with light having a wavelength or waveband corresponding to a characteristic absorption waveband of the photosensitive compound, such as about 500 or about 600 nm to about 800 nm or about 1100 nm, which activates the photosensitive compound. Activating the photosensitive compound may cause singlet oxygen radicals and other reactive species to be generated, leading to a number of biological effects that may destroy the tissue which has absorbed the photosensitive compound such as abnormal or diseased tissue.

The photosensitive compound may be any compound or pharmaceutically acceptable salts or hydrates thereof, which react as a direct or indirect result of absorption of ultraviolet, visible, or infrared light. In one embodiment, the photosensitive compound reacts as a direct or indirect result of absorption of red light. The photosensitive compound may be a compound which is not naturally in the tissue. Alternatively, the photosensitive compound may naturally be present in the tissue, but an additional amount of the photosensitive compound may be administered to the mammal. In some embodiments, the photosensitive compound may selectively bind to one or more types of selected target cells and, when exposed to light of an appropriate waveband, absorb the light, causing substances to be produced that impair or destroy the target cells.

While not limiting any embodiment, for some types of therapies, it may be helpful if the photosensitive compound is nontoxic to the animal to which it is administered or is capable of being formulated in a nontoxic composition that can be administered to the animal. In some embodiments, it may also be helpful if the photodegradation products of the photosensitive compounds are nontoxic.

Some non-limiting examples of photosensitive chemicals may be found in Kreimer-Bimbaum, Sem. Hematol, 26:157-73, (1989), incorporated by reference herein in its entirety, and include, but are not limited to, chlorins, e.g., Tetrahydroxylphenyl chlorin (THPC) [652 nm], bacteriochlorins [765 nm], e.g., N-Aspartyl chlorin e6 [664 nm], phthalocyanines [600-700 nm], porphyrins, e.g., hematoporphyrin [HPD][630 nm], purpurins, e.g., [1,2,4-Trihydroxyanthraquinone] Tin Etiopurpurin [660 nm], merocyanines, psoralens, benzoporphyrin derivatives (BPD), e.g., verteporfin, and porfimer sodium; and pro-drugs such as delta-aminolevulinic acid or methyl aminolevulinate, which can produce photosensitive agents such as protoporphyrin IX. Other suitable photosensitive compounds include indocyanine green (ICG) [800 nm], methylene blue [668 nm, 609 nm], toluidine blue, texaphyrins, Talaportin Sodium (mono-L-aspartyl chlorine)[664 nm], verteprofin [693 nm], which may be useful for phototherapy treatment of conditions such as age-related macular degeneration, ocular histoplasmosis, or pathologic myopia], lutetium texaphyrin [732 nm], and rostaporfin [664 nm].

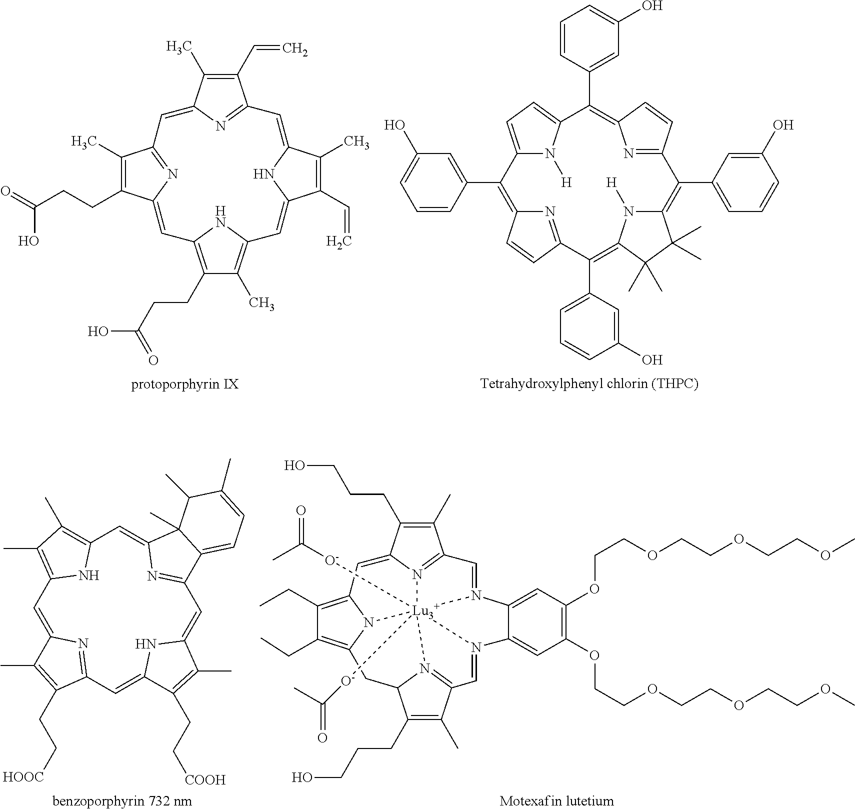

In some embodiments, the photosensitive compound comprises at least one component of porfimer sodium. Porfimer sodium comprises a mixture of oligomers formed by ether and ester linkages of up to eight porphorin units. The structural formula below is representative of some of the compounds present in porfimer, wherein n is 0, 1, 2, 3, 4, 5, or 6 and each R is independently —CH(OH)CH₃ or —CH═CH₂.

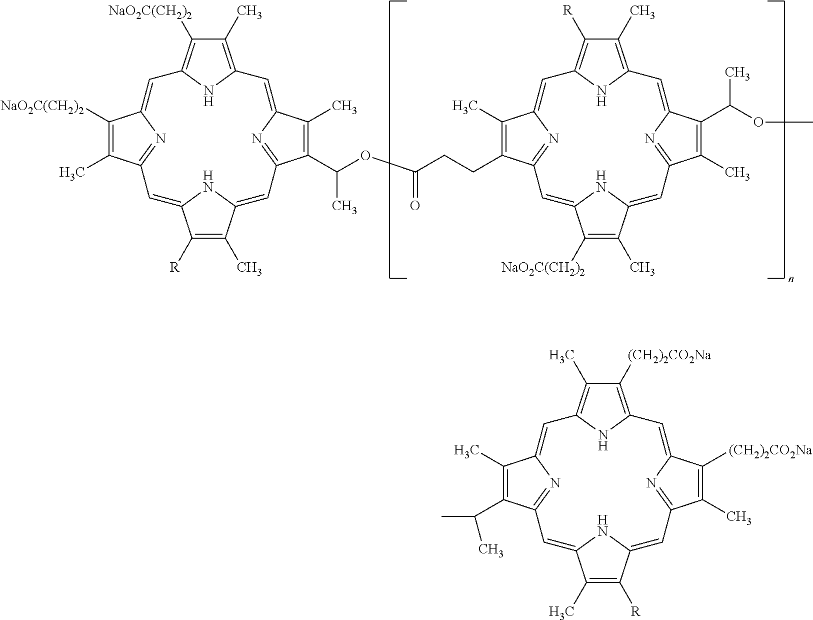

In some embodiments, the photosensitive compound is at least one of the regioisomers of verteporphin, shown below.

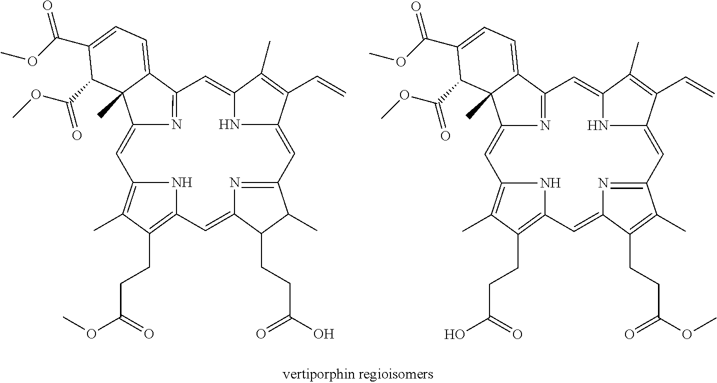

In some embodiments, the photosensitive compound comprises a metal analogue of phthalocyanine shown below.

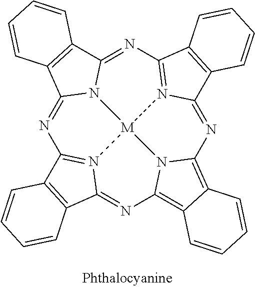

In one embodiment, M is zinc. In one embodiment, the compound can be zinc phthalocyanine or zinc phthalocyanine tetrasulfonate.

A photosensitive agent can be administered in a dry formulation, such as a pill, a capsule, a suppository or a patch. The photosensitive agent may also be administered in a liquid formulation, either alone, with water, or with pharmaceutically acceptable excipients, such as those disclosed in Remington's Pharmaceutical Sciences. The liquid formulation also can be a suspension or an emulsion. Liposomal or lipophilic formulations may be desirable. If suspensions or emulsions are utilized, suitable excipients may include water, saline, dextrose, glycerol, and the like. These compositions may contain minor amounts of nontoxic auxiliary substances such as wetting or emulsifying agents, antioxidants, pH buffering agents, and the like. The above described formulations may be administered by methods which may include but are not limited to intradermal, intramuscular, intraperitoneal, intravenous, subcutaneous, intranasal, epidural, oral, sublingual, intranasal, intracerebral, intravaginal, transdermal, iontophoretical, rectally, by inhalation, or topically to the desired target area, for example, the body cavity (oral, nasal, rectal), ears, nose, eyes, or skin. The preferred mode of administration is left to the discretion of the practitioner, and will depend in-part upon the site of the medical condition (such as the site of cancer or viral infection).

The dose of photosensitive agent may vary. For example, the target tissue, cells, or composition, the optimal blood level, the animal's weight, and the timing and duration of the radiation administered, may affect the amount of photosensitive agent used. Depending on the photosensitive agent used, an equivalent optimal therapeutic level may have to be empirically established. The dose may be calculated to obtain a desired blood level of the photosensitive agent, which in some embodiments may be from about 0.001 g/mL or about 0.01 μg/ml to about 100 μg/ml or about 1000 μg/ml.

In some embodiments, about 0.05 mg/kg or about 1 mg/kg to about 50 mg/kg or about 100 mg/kg is administered to the mammal. Alternatively, for topical application, about 0.15 mg/m² or about 5 mg/m² to about 30 mg/m² or about 50 mg/m² may be administered to the surface of the tissue.

The light may be administered by an external or an internal light source, such as an OLED device described herein. The intensity of radiation or light used to treat the target cell or target tissue may vary. In some embodiments, the intensity may be about 0.1 mW/cm² to about 100 mW/cm², about 1 mW/cm² to about 50 mW/cm², or about 3 mW/cm² to about 30 mW/cm². The duration of radiation or light exposure administered to a subject may vary. In some embodiments the exposure ranges from about 1 minute, about 60 minutes, or about 2 hours to about 24 hours, about 48 hours, or about 72 hours.

A certain amount of light energy may be required to provide a therapeutic effect. For example, a certain amount of light energy may be required to activate the photosensitive compounds. This may be accomplished by using a higher power light source, which may provide the needed energy in a shorter period of time, or a lower power light source may be used for a longer period of time. Thus, a longer exposure to the light may allow a lower power light source to be used, while a higher power light source may allow the treatment to be done in a shorter time. In some embodiments, the total fluence or light energy administered during a treatment may be in the range of about 5 Joules to about 1,000 Joules, about 20 Joules to about 750 Joules, or about 50 Joules to about 500 Joules.

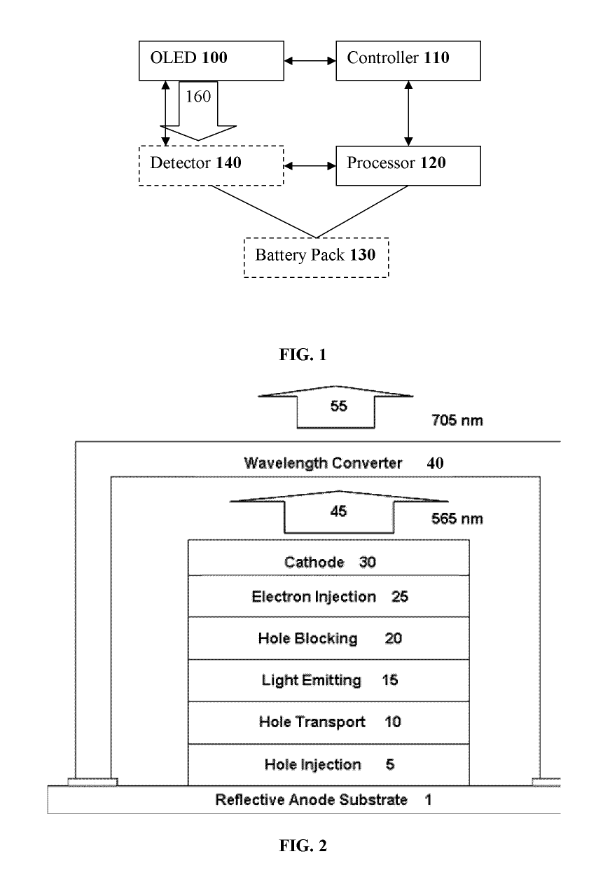

FIG. 1 is a schematic of some embodiments which further include a controller 110 and processor 120 electrically connected to an organic light-emitting diode 100 (OLED), which may help to provide a uniform power supply to facilitate homogeneous light exposure of the tissue. In some embodiments, the apparatus further includes an optional detector 140, such as photodiode, which detects a portion of the light 160 emitted from the OLED 100, to help determine the amount of light being emitted by the OLED 100. For example, the detector 140 may communicate a signal related to the intensity of the light 160 received from the OLED 100 to the processor 120, which, based upon the signal received, may communicate any desired power output information to the controller 100. Thus, these embodiments may provide real time feedback which allows the control of the intensity of light emitted from the OLED 100. The detector 140 and the processor 120 may be powered by compact power supply, such as a battery pack 130, or by some other power source.

In some embodiments, the device may further include a wireless transmitter electrically connected to an component of the apparatus generating treatment information, e.g., level of intensity, time of application, dosage amount, to communicate/transfer data to another external receiving device, like cell phone, PDA or to doctor's office. In some embodiments, the apparatus may further include an adhesive tape which may be used to attach the apparatus on the tissue surface so as to stabilize it on the target area.

For phototherapy and other applications, a wavelength convertor may be positioned in the device to receive at least a portion of light emitted from the organic light-emitting diode in a lower wavelength range, such as about 350 nm to less than about 600 nm, and convert at least a portion of the light received to light in a higher wavelength range, such as about 600 nm to about 800 nm. The wavelength convertor may be a powder, a film, a plate, or in some other form and, may comprise: yttrium aluminum garnet (YAG), alumina (Al₂O₃), yttria (Y₂O₃), titania (TiO₂), and the like. In some embodiments, the wavelength convertor may comprise at least one dopant which is an atom or an ion of an element such as Cr, Ce, Gd, La, Tb, Pr, Sm, Eu, etc.

In some embodiments, translucent ceramic phosphor is represented by a formula such as, but not limited to (A₁₋ₓEₓ)₃D₅O₁₂, (Y₁₋ₓEₓ)₃D₅O₁₂; (Gd₁₋ₓEₓ)₃D₅O₁₂; (La₁₋ₓEₓ)₃D₅O₁₂; (Lu₁₋ₓEₓ)₃D₅O₁₂; (Tb₁₋ₓEₓ)₃D₅O₁₂; (A₁₋ₓEₓ)₃Al₅O₁₂; (A₁₋ₓEₓ)₃Ga₅O₁₂; (A₁₋ₓEₓ)₃In₅O₁₂; (A₁₋ₓCeₓ)₃D₅O₁₂; (A₁₋ₓEₓ)₃D₅O₁₂; (A₁₋ₓTbₓ)₃D₅O₁₂; (A₁₋ₓEₓ)₃Nd₅O₁₂; and the like. In some embodiments, the ceramic comprises a garnet, such as a yttrium aluminum garnet, with a dopant. Some embodiments provide a composition represented by the formula (Y₁₋ₓCeₓ)₃Al₅O₁₂. In any of the above formulas, A may be Y, Gd, La, Lu, Tb, or a combination thereof; D may be Al, Ga, In, or a combination thereof; E may be Ce, Eu, Tb, Nd, or a combination thereof; and x may be in the range of about 0.0001 to about 0.1, from about 0.0001 to about 0.05, or alternatively, from about 0.01 to about 0.03.

In some embodiments, the apparatus is a top emitting device, wherein the OLED is mounted upon a non-emissive substrate, and a wavelength convertor is mounted above the top layer of the light-emitting diode. In some embodiments, the substrate and the wavelength converting layer may cooperate with each other to provide a protective seal such as a moisture seal.

A non-limiting example of such a device is depicted in FIG. 2. These embodiments may comprise a reflective anode 1, which may act as a substrate for the device. A hole injection layer 5, if present, may be disposed on the anode 1. A hole-transport layer 10, if present, may be disposed on the hole injection layer 5. A light-emitting layer 15 may be disposed on the hole-transport layer 10. A hole-blocking layer 20, if present, may be disposed on the light-emitting layer 15. An electron-injection layer 25, if present, may be disposed on the hole-blocking layer 20. A cathode 30 may be disposed on the electron-injection layer 25. Finally, a wavelength convertor 40 may encapsulate the entire device, such as in the manner shown. In these embodiments, the wavelength convertor 40 absorbs visible light 45 emitted from the OLED and emits near IR light 55. For the particular layers described above, yellow light 45 from the OLED having an average wavelength of about 565 nm was absorbed by a Cr co-doped YAG plate wavelength convertor 40, which emitted near IR light 55 having an average wavelength of about 705 nm. Here, Cr:YAG was chosen because it absorbs strongly at 565 nm and strongly emits Near-IR.

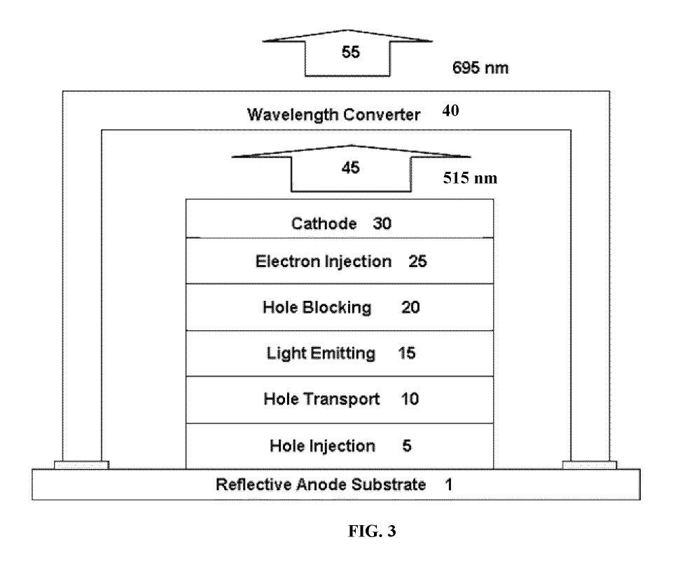

The character of the light absorbed or emitted by a wavelength convertor may vary. For example, in the embodiment depicted in FIG. 3, the wavelength convertor 40 absorbs visible light 45 emitted from the OLED and emits near IR light 55. For example, yellow light 45 from the OLED having an average wavelength of about 515 nm may be absorbed by a Cr doped alumina wavelength convertor 40, which may emit near IR light 55 having an average wavelength of about 695 nm.

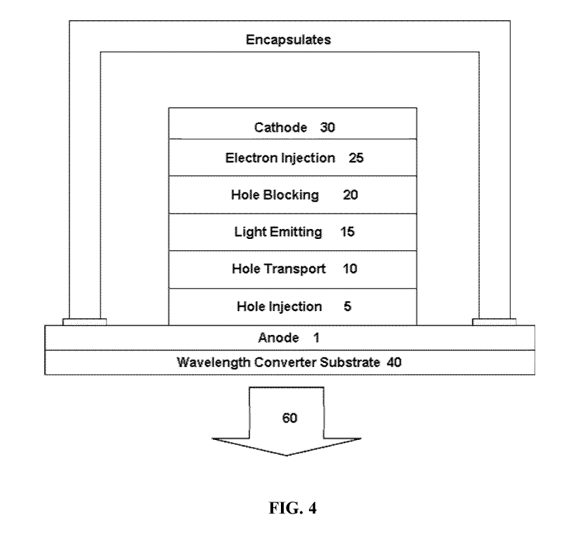

In some embodiments, the apparatus may be a bottom emitting device, wherein the OLED is mounted to the wavelength converting layer. For example, as shown in FIG. 4, a wavelength converting layer 40 may serve as both a substrate and wavelength conversion layer. An anode 1 may be disposed on the wavelength convertor 40 substrate. A hole injection layer 5, if present, may be disposed in the anode layer 1. A hole-transport layer 10, if present, may be disposed on the hole injection layer 5. A light-emitting layer 15 may be disposed on the hole-transport layer 10. A hole-blocking layer 20, if present, may be disposed on the light-emitting layer 15. An electron-injection layer 25, if present, may be disposed in the hole-blocking layer 20. Finally, a cathode 30 may be disposed on the electron-injection layer 25. The entire device may be encapsulated by a barrier material 70, to protect the device from oxygen and moisture.

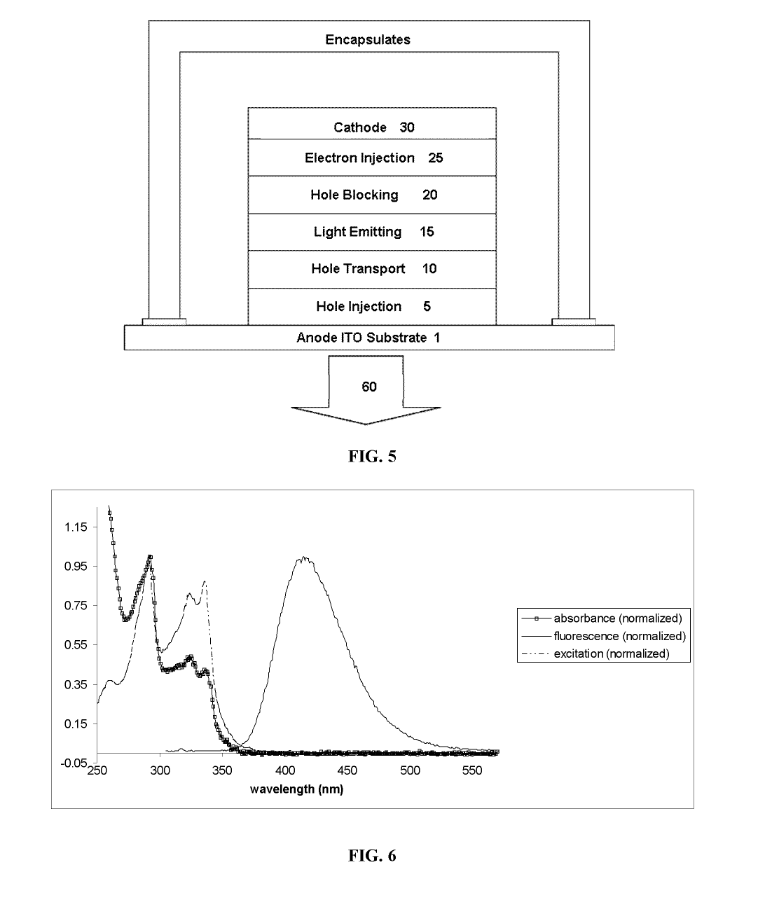

In some embodiments, the apparatus is a bottom emitting device, wherein the OLED is fabricated on the conventional glass-ITO substrate. For example, as shown in FIG. 5, an anode 1 may used on glass substrate. A hole injection layer 5, if present, may be disposed in the anode layer 1. A hole-transport layer 10, if present, may be disposed on the hole injection layer 5. A light-emitting layer 15 may be disposed on the hole-transport layer 10. A hole-blocking layer 20, if present, may be disposed on the light-emitting layer 15. An electron-injection layer 25, if present, may be disposed in the hole-blocking layer 20. Finally, a cathode 30 may be disposed on the electron-injection layer 25. The entire device may be encapsulated by a barrier material 70, to protect the device from oxygen and moisture.

### Example 1

An example of the host compound may be synthesized according to the following scheme:

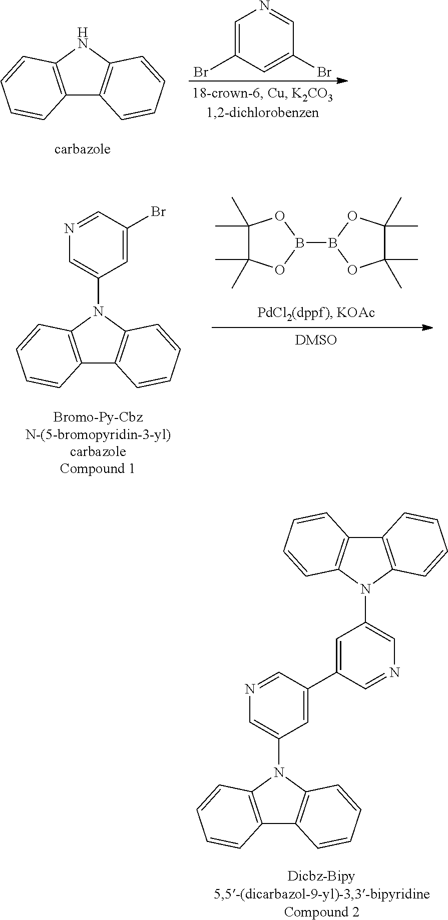

### Experimental

Bromo-Py-Cbz (1). Compound 1 was made according to a procedure adapted from Hou, Z.; Liu, Y.; Nishiura, M.; Wang, Y. J. Am. Chem. Soc. 2006, 128(17), 5592-5593. A mixture of carbazole (4.751 g, 28.41 mmol), 3,5-dibromopyridine (20.19 g, 85.23 mmol), K₂CO₃ (15.71 g, 113.6 mmol), copper powder (1.204 g, 18.94 mmol), 18-crown-6 ether (2.503 g, 9.470 mmol) and 1,2-dichlorobenzene (150 mL) was degassed with argon for about 1 h while stirring. The reaction mixture was then maintained at about 200° C. with stirring under argon for about 20 h. Upon cooling to room temperature (RT), the crude mixture was filtered and concentrated in vacuo. The resulting residue was then purified by flash chromatography (SiO₂, 1:1 to 11:9 dichloromethane-hexanes) to afford 1 (6.75 g, 74%) as a white solid: mp=118-120° C.; ¹H NMR (400 MHz, CDCl₃): δ 8.81 (dd, J=23.2, 2.0 Hz, 2H), 8.14 (d, J=7.7 Hz, 2H), 8.10 (t, J=2.2 Hz, 1H), 7.47-7.32 (m, 6H); ¹³C NMR (100.5 MHz, CDCl₃): δ 149.4, 146.5, 140.2, 136.8, 135.4, 126.4, 123.8, 121.0, 120.9, 120.6, 109.2.

Dicbz-Bipy (2). A mixture of 1 (1.500 g, 4.641 mmol), bis(pinacolato)diboron (0.648 g, 2.55 mmol), [1,1′-bis(diphenylphosphino)-ferrocene]dichloropalladium(II) (114 mg, 0.139 mmol), potassium acetate (1.367, 13.92 mmol) and DMSO (38 mL) was degassed with argon for about 30 min while stirring. The reaction mixture was then maintained at about 90° C. with stirring under argon for about 46 h. Upon cooling to RT, the reaction was poured over dichloromethane (250 mL) and the organics washed with sat. NaHCO₃, water (4×) and brine. The organic phase was then dried over MgSO₄, filtered and concentrated in vacuo. Purification of the crude product by flash chromatography (SiO₂, 49:1 dichloromethane-acetone) and subsequent recrystallization from hexanes and dichloromethane (ca. 2:1) yielded 2 (1.09 g, 96%) as an off-white solid: mp=239-241° C.; ¹H NMR (400 MHz, CDCl₃): δ 9.01 (dd, J=11.0, 2.2 Hz, 4H), 8.21 (t, J=2.2 Hz, 2H), 8.17 (d, J=7.7 Hz, 4H), 7.45 (d, J=3.3 Hz, 8H), 7.36-7.32 (m, 4H); ¹³C NMR (100.5 MHz, CDCl₃): δ 148.4, 146.6, 140.4, 135.1, 133.7, 132.6, 126.4, 123.8, 120.9, 120.6, 109.2; Anal. Calcd. for C₃₄H₂₂N₄: C, 83.93; H, 4.56; N, 11.51. Found: C, 83.43; H, 4.57; N, 11.32.

The spectroscopic properties of 2 in chloroform were obtained, and the results are depicted in FIG. 6.

### Example 2

Device A was fabricated as follows. A glass-ITO substrate having sheet resistance of about 20 ohm/sq was sequentially cleaned by detergent, water, isopropyl alcohol (IPA) and acetone with ultra-sonication followed by UV ozone treatment for about 30 min. The substrate was then transferred into a vacuum chamber for deposition of different layers. A reflective anode as silver (Ag) was deposited at a rate of about 0.3 nm/s for about 100 nm thickness. A hole injection layer as MoO₃ was deposited at a rate of 0.05 nm/s for about 10 nm. Then a hole transporting layer such as N,N′-Di(napth-1-yi)-N,N′-diphenyl-benzidine (NPD) was deposited at a rate of about 0.1 nm/s for about 40 nm. The emissive material, fac tris(2-phenylpyridine) iridium (Ir(ppy)₃)(9 wt %) was co-deposited with one bipolar host material 5,5-(dicarbazol-9-yl)-3,3′-bipyridine (Compound 2) at about 0.01 nm/sec and about 0.1 nm/s respectively to make the appropriate thickness ratio. A hole blocking layer of 1,3,5-Tris(1-phenyl-1H-benzimidazol-)-2-yl)benzene (TPBI) was then deposited at about 0.1 nm/sec rate on the emissive layer. A very thin layer of electron injection, lithium fluoride (LiF), was deposited at about 0.005 nm/s rate and a thin layer of aluminum (Al) was vacuum deposited at about 0.005 nm/sec. Finally a semi-transparent silver layer was deposited at about 0.1 nm/sec. All the materials were deposited at a vacuum level of about 5×10⁻⁷ torr. The total device structure can be represented as ITO(150 nm)/Ag(100 nm)/MoO₃(10 nm)/NPD (40 nm)/Compound 2:Ir(ppy)₃ (30 nm)/TPBI (30 nm)/LiF(0.5 nm)/Al(2 nm)/Ag(15 nm). Total thickness of the device varied from about 100 to about 200 nm (electrode-to-electrode). All spectra were measured with a Spectrascan spectroradiometer PR-670 (Photo Research, Inc., Chatsworth, Calif., USA); and I-V-L characteristics were taken with a Keithley 2612 SourceMeter (Keithley Instruments, Inc., Cleveland, Ohio, USA) and PR-670. All device operation was performed inside a nitrogen-filled glove-box

### Example 3

Device B was fabricated as follows. A glass-ITO substrate having sheet resistance of about 20 ohm/sq was sequentially cleaned by detergent, water, isopropyl alcohol (IPA) and acetone with ultra-sonication and followed by UV ozone treatment for about 30 minutes. A hole injection layer as PEDOT:PSS was spin coated on the substrate at about 5000 rpm for about 30 sec in order to have a thickness of about 40 nm. The substrate was baked at about 100° C. for about 30 min in a normal environment (air) followed by baking at about 200° C. for about 30 min inside a glove box and N₂ environment in order to remove any trace amount of solvent. The substrate is then transferred into a vacuum chamber, where hole transporting layer such as N,N′-Di(napth-1-yl)-N,N′-diphenyl-benzidine (NPD) was vacuum deposited at a rate of about 0.1 nm/s. The emissive material, Bis[(1-phenylisoquinolinato-N,C2′)]iridium (III) (acetylacetonate) (Ir(piq)₂acac)(9 wt %) was then co-deposited with one bipolar host material 5,5-(dicarbazol-9-yl)-3,3′-bipyridine (Compound 2) at about 0.01 nm/sec and about 0.1 nm/s respectively to make the appropriate thickness ratio. A hole blocking layer of 1,3,5-Tris(1-phenyl-1H-benzimidazol-)-2-yl)benzene (TPBI) was then deposited at the rate of about 0.1 nm/sec on the emissive layer. A very thin layer of electron injection layer, lithium fluoride (LiF) was then deposited at the rate of about 0.005 nm/s and the aluminum (Al) cathode was vacuum deposited at about 0.3 nm/sec. All the materials were deposited at a vacuum level of about 5×10⁻⁷ torr. The total device structure can be represented as ITO(150 nm)/PEDOT:PSS(40 nm)/NPD(40 nm)/Compound 2:Ir(piq)₂acac (30 nm)/TPBI (30 nm)/LiF(0.5 nm)/Al(120 nm). Total thickness of the device varied from about 100-150 nm (electrode-to-electrode).

### Example 4

Device C was fabricated in the same manner as Device A in Example 2, except that Ir(pthpy)₂acac was used instead of Ir(ppy)₃. Electroluminescence (EL) spectrum of Devices A, B and C are shown in FIG. 8. In FIG. 8, Device A is shown as having an emission peak at about 515 nm, Device B, is shown as having an emission peak at about 630 nm, and Device C is shown as having an emission peak at about 565 nm. In all cases, bipolar Compound 2 exhibits a complete energy transfer to the emissive dopants with about 9 wt % doping concentration. No residual shoulder emission supports host-guest energy transfer process, indicating a good charge balance in the device with bipolar host materials.

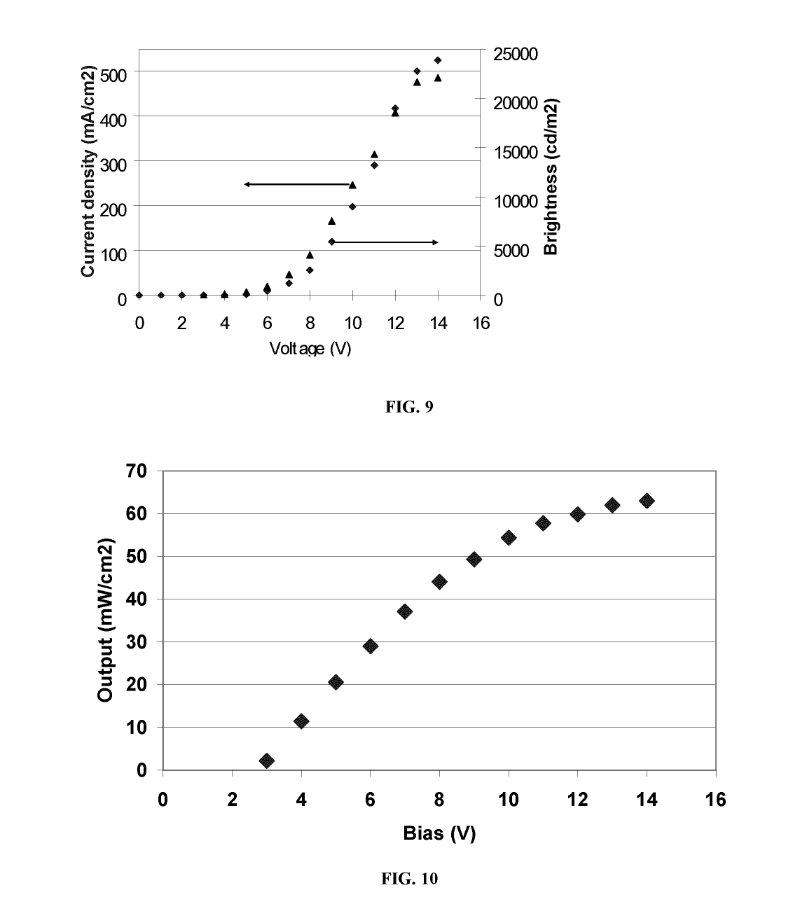

In addition, device performance of Device B was evaluated by measuring the current density and luminance as a function of the driving voltage, as shown in FIG. 9. The turn-on voltage for Device B was about 2.6 volts and the maximum luminance was about 15000 cd/m². The EQE of Device B was about 22%, and the luminous efficiency was about 16 cd/A at 1000 cd/m². The power efficiency (PE) was 14 lm/w at 1000 cd/m². These values are similar to some conventional OLED devices. FIG. 10 shows the optical output power (mW/cm²) as a function of applied voltage. For some currently practiced photodynamic therapy applications, an optical power output of about 10-150 mW/cm² may be helpful to provide a sufficient light dose to the patient within a reasonable amount of time. Thus, use of this device should be useful for photodynamic therapy applications.

### Example 5

### Example of Measuring Charge Mobility from Space Charge Limited Current (SCLC) Regime

The usefulness of the compound disclosed herein is demonstrated by their charge mobility. The carrier mobility of an organic thin film can be derived from the space charge limited current in the current-voltage (IV) measurement based on the Mott's steady state SCLC model

$$J = \frac{9 {ɛɛ}_{0} μ {V}^{2}}{8 {L}^{3}}$$

where ∈₀ is the vacuum permittivity, ∈ is the relative permittivity of the organic layer, μ is the carrier mobility of the organic layer, V is the voltage bias and L is the thickness of the organic layer.

To evaluate the electron and hole mobility of an organic layer, single-carrier devices (electron-only and hole-only devices) may be made. Electron-only devices may have Al/organic layer/LiF/Al structure with Al as the anode and LiF/Al as the cathode. The LiF/Al electrode has a low work function (˜2.6 eV) which can facilitate the injection of electrons into the lower lying LUMO of the organic layer. By contrast, Al has a relatively lower work function (4.28 eV) than the HOMO (5˜6 eV) of the organic layer being investigated, which prevents the hole injection from the anode. Thus, only electrons are injected into the organic layer and the electron mobility may be measured as the only charge carrier in the organic layer.

The hole-only devices may have the ITO/PEDOT/organic layer/Al with ITO as the anode and Al as the cathode. The high work function of PEDOT (5.2-5.4 eV) facilitates hole injection from the anode into the organic layer. By contrast, the work function (4.28 eV) of Al is higher than the LUMO of the organic layer (2˜4 eV), which preventing the electron injection from the cathode. Thus, only holes are injected into the organic layer, and the hole mobility may be measured as the only charge carrier in the organic layer.

The thickness of the organic layer is kept at 100 nm in both cases.

To measure the space charge limited current, one applies a large voltage scan (0-10 V) on the device to ensure at large current limit the device is under SCLC condition. And then the IV curve is fitted by the SCLC model mentioned above. The carrier mobility can then be derived from the fitting parameters. Electron- and hole-mobility can be derived from the electron-only (Device D) and hole-only (Device E) devices for the same organic layer, respectively.

### Example 6

Fabrication for single-carrier devices (hole only device) (Device D): the substrates (ITO coated glass) having sheet resistance of about 20 ohm/sq was sequentially cleaned by detergent, water, isopropyl alcohol (IPA) and acetone with ultra-sonication and followed by UV ozone treatment for about 30 minutes. A hole injection layer as PEDOT:PSS was spin coated on the substrate at about 5000 rpm for about 30 sec yielding a thickness of around 40 nm. The substrate was baked at about 100° C. for about 30 min in a normal environment (air) followed by baking at about 200° C. for about 30 min inside a glove box and N₂ environment in order to remove any trace amount of solvent. The substrate was then transferred into a vacuum chamber, where the organic layer (compound 2) was vacuum deposited at a rate of about 0.1 nm/s rate yielding a thickness about 100 nm. A 120 nm-thick Al layer was then deposited successively by thermal evaporation at deposition rate of about 0.3 nm/s, through a shadow mask to define the device area. All the materials were deposited at a vacuum level of about 5×10⁻⁷ torr.

Fabrication for single-carrier devices (electron only device) (Device E): the substrates (glass only) was sequentially cleaned by detergent, water, isopropyl alcohol (IPA) and acetone with ultra-sonication and followed by UV ozone treatment for 30 minutes. The substrate was then transferred into a vacuum chamber. About 20 nm of Al was deposited as bottom electrode at a rate of about 0.1 nm/s through a shadow mask. Then an organic layer (compound 2) was vacuum deposited at a rate of about 0.1 nm/s rate yielding a thickness about 100 nm. The top electrode LiF and Al were then deposited successively through a shadow mask, at deposition rates of 0.05 and 0.3 nm/s to achieve the thickness of 0 about 5 nm and about 120 nm, respectively.

The device areas for hole-only and electron-only devices are 0.08 and 0.04 cm², respectively. I-V measurements were carried out using a Keithley 2400 Source Meter to apply 0-10 V voltage scans and measure the current simultaneously. All device operations were done inside a nitrogen-filled glove-box. The high current end of the I-V curves (6-10 V) were fitted by the SCLC model

$$J = \frac{9 {ɛɛ}_{0} μ {V}^{2}}{8 {L}^{3}} .$$

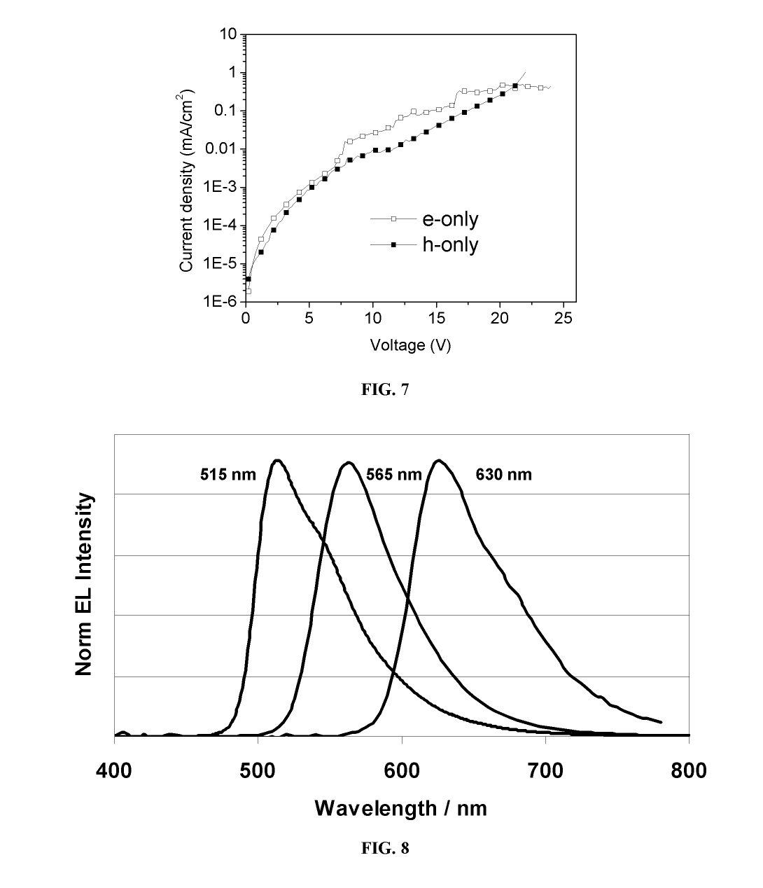

The electron- and hole-mobility can then be derived from the fitting parameters for the electron-only and hole-only devices, respectively. The mobility values of Compound 2 obtained by SCLC model are 1.4×10⁻⁸ cm²/V-s (hole only) and 2.4×10⁻⁸ cm²/V-s (electron only). The dielectric constant of the film was estimated as 2.18. The mobility values concludes that the host material Compound 2 is a bipolar material where the transport of hole and electron can be balanced precisely to achieve higher efficiency OLED. The I-V spectra of Device D and Device E are shown in FIG. 7, showing balanced hole-current and electron-current, indicating Compound 2 has ambipolar property. Note that there is no any detectable electroluminescence (EL) during the measurements in each device, ensuring uni-polar injection.

### Example 7

An example of an embodiment of a device depicted in FIG. 2 was fabricated in a manner similar to that of Device C of example 4, where the successive layers were deposited on the reflective anode substrate and the device was encapsulated with a wavelength convertor layer. The wavelength convertor layer can be prepared in the following manner.

A YAG:Cr wavelength convertor was then fabricated as follows. A 50 ml high purity Al₂O₃ ball mill jar was filled with 55 g of Y₂O₃-stabilized ZrO₂ ball of 3 mm diameter. Then, in a 20 ml glass vial, 0.153 g dispersant (Flowlen G-700. Kyoeisha), 2 ml xylene (Fisher Scientific, Laboratory grade) and 2 ml ethanol (Fisher Scientific, reagent alcohol) were mixed until the dispersant was dissolved completely. The dispersant solution and tetraethoxysilane as sintering aid (0.038 g, Fluka) were added to a ball mill jar. Y₂O₃ powder (3.984 g, 99.99%, lot N-YT4CP, Nippon Yttrium Company Ltd.) with a BET surface area of 4.6 m²/g and Al₂O₃ powder (2.968 g, 99.99%, grade AKP-30, Sumitomo Chemicals Company Ltd.) with a BET surface area of 6.6 m²/g and 0.118 g Chromium (III) nitrate nonahydrate (99.99% pure, Sigma-Aldrich) were then added to a ball mill jar. The total powder weight was 7.07 g and the ratio of Y₂O₃ to Al₂O₃ was at a stoichiometric ratio of 3:5. A first slurry was produced by mixing the Y₂O₃ powder, the Al₂O₃ powder and chromium nitrate, dispersant, tetraethoxysilane, xylenes, and ethanol by ball milling for 24 hours.

A solution of binder and plasticizers was prepared by dissolving 3.5 g poly(vinyl butyral-co-vinyl alcohol-co-vinyl acetate) (Aldrich), 1.8 g benzyl n-butyl phthalate (98%, Alfa Aesar), and 1.8 g polyethylene glycol (Mn=400, Aldrich) in 12 ml xylene (Fisher Scientific, Laboratory grade) and 12 ml ethanol (Fisher Scientific, reagent alcohol). A second slurry was produced by adding 4 g of the binder solution into the first slurry and then milling for another 24 hours. When ball milling was complete, the second slurry was passed through a syringe-aided metal screen filter with pore size of 0.05 mm. Viscosity of second slurry was adjusted to 400 centipoise (cP) by evaporating solvents in the slurry while stirring at room temperature. The slurry was then cast on a releasing substrate, e.g., silicone coated Mylar® carrier substrate (Tape Casting Warehouse) with an adjustable film applicator (Paul N. Gardner Company, Inc.) at a cast rate of 30 cm/min. The blade gap on the film applicator was set at 0.38 mm (15 mil). The cast tape was dried overnight at ambient atmosphere to produce a green sheet of about 95 μm thickness. Finally, the green sheet was peeled off from substrate and cut into sheets of 10 cm×10 cm size.

The green sheets (e.g four) thus obtained were piled up and constituted onto carrier substrate, followed by 90° C.-heated compression in a hydraulic press at a uniaxial pressure of 8 metric tons and held at that pressure for 5 minutes. Laminated composites of four emissive layers were thus produced. The carrier substrate with silicone releasing coating was carefully removed from laminated green sheets. Any number of green sheets can be laminated using this method.

For debindering, laminated green sheets were sandwiched between ZrO₂ cover plates (1 mm in thickness, grade 42510-X, ESL Electroscience Inc.) and placed on an Al₂O₃ plate of 5 mm thick; then heated in a tube furnace in air at a ramp rate of 0.5° C./min to 600° C. and held for 2 hours to remove the organic components from the green sheets to generate a preform.

After debindering, the preforms were annealed at 1500° C. in a vacuum of 10⁻¹ Torr for 5 hours at a heating rate of 1° C./min. Following the first annealing, the preforms were further sintered in a vacuum of 10⁻³ Torr at about 1650° C. for 2 hours at a heating rate of 5° C./min and a cooling rate of 10° C./min to room temperature to produce a translucent ceramic sheet of about 0.38 mm thickness. Sintered ceramic sheets were reoxidized in a furnace under vacuum of 10⁻¹ Torr at 1400° C. for about 2 hrs at heating and cooling rates of 10° C./min and 20° C./min respectively. After annealing, the preforms were diced (MTI Corp, EC-400 Precision CNC dicing) to about 20 mm×15 mm blocks.

The preformed sheets can be made in different shapes such as square, rectangular, circular etc. Alternatively the preforms can be made in dome shaped or in a rectangular cuvette shape by using appropriate metal mold.

The device is then encapsulated with a wavelength convertor, such as a specific plate or film or embedded structure prepared by the method described above. Yellow light from the OLED having an average wavelength of about 565 nm was absorbed by a Cr co-doped YAG plate wavelength convertor, which emitted near IR light having an average wavelength of about 705 nm. Here, Cr:YAG was chosen because it absorbs strongly at 565 nm and strongly emits Near-IR.

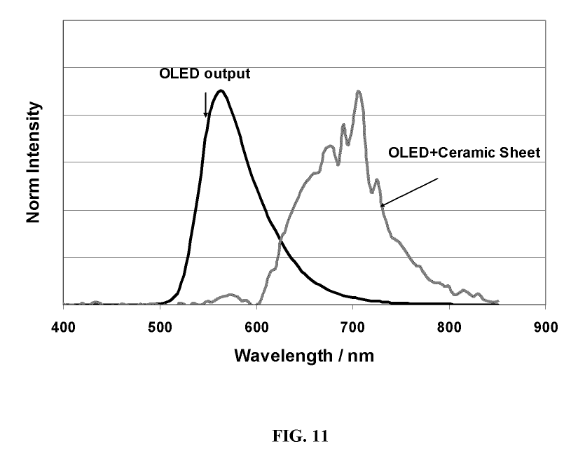

FIG. 11 exhibits normalized spectra of an OLED without a wavelength convertor and an OLED with wavelength converter. As shown in FIG. 11, the emission of the device without the wavelength convertor has an average of about 565 nm, and the emission with the wavelength convertor has an average of about 705 nm.

### Example 8

An example of a device structured as shown in FIG. 3, was prepared in a manner similar to Device A of Example 2. In this example, yellow light from the OLED having an average wavelength of about 515 nm was absorbed by a Cr doped alumina wavelength convertor, which emitted near IR light having an average wavelength of about 695 nm. The device is then encapsulated with a wavelength convertor (Cr:Al₂O₃), such as a specific plate or film prepared by the method described above.

The Cr:Al₂O₃ wavelength convertor was fabricated in a similar manner to the Cr:YAG wavelength convertor except that Al₂O₃ powder (5.936 g, 99.99%, grade AKP-30, Sumitomo Chemicals Company Ltd.) with a BET surface area of 6.6 m²/g and 0.235 g Chromium (III) nitrate nonahydrate (99.99% pure, Sigma-Aldrich) were added to ball mill jar after the disperant solution and sintering aid instead of Y₂O₃ powder (3.984 g), Al₂O₃ powder (2.968 g) and 0.118 g Chromium (III) nitrate non-anhydrate.

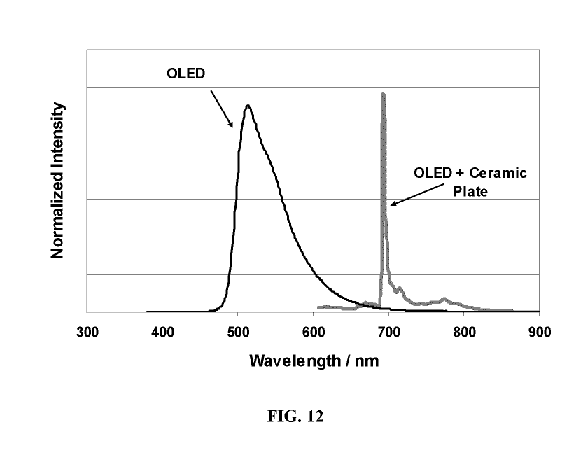

FIG. 12 exhibits normalized spectra of OLED only and the integrated device. As shown in FIG. 12, the OLED without the wavelength convertor has broad emission with a maximum at around about 515 nm, but the OLED with the Cr:Alumina wavelength convertor shows a very sharp emission with a narrow full width at half maximum. Such a narrow emission may be advantageous for PDT applications.

### Example 9

An example of an integrated device as depicted in FIG. 4 is fabricated in an additive process. A ceramic plate (Cr:Al₂O₃ or (Cr:YAG), with a 30 mm×30 mm size is cleaned by the usual way as described above and then brought into a deposition chamber. A 50 um transparent photo resist layer can be disposed and cured on the ceramic plate in order to planarize the surface. A transparent anode typically indium tin oxide (ITO) or indium zinc oxide (IZO) having 100 nm thickness can be sputtered, or a thin silver (Ag) with 20 nm thickness can be deposited with an appropriate mask. Then a hole injection layer e.g. MoO3 co-doped with NPD with a thickness of 40 nm on the anode is deposited. Next, an electron blocking-hole transporting layer NPD with thickness of 10 nm is deposited. A light emitting layer as Compound 2 is co-deposited with Ir(ppy)₃ or Ir(pthpy)₂acac (9 wt %) (30 nm), followed by a hole blocking layer of TPBI (30 nm). Finally an electron injection layer as LiF(0.5 nm) and cathode Al (120 nm) is deposited. The device is then encapsulated with a glass cap and epoxy adhesive to protect from moisture and oxygen. As mentioned earlier that the integrated device structure in the present invention can convert about 60% of visible light to near-IR, and can thus provide 20-30 mW/cm2 output power. This output is quite significant for an OLED at such a long wavelength, because a conventional OLED structure's efficiency decreases with increasing wavelength due to the energy gap law. Thus, such as device may treat deeper tissue and achieve higher efficacy.

### Example 10

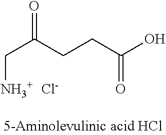

5-Aminolevulinic acid HCl (20% topical solution, available as LEVULAN® KERASTICK® from DUSA® Pharmaceuticals) is topically applied to individual lesions on a person suffering from actinic keratoses. About 14-18 hours after application, the treated lesions are illuminated with a red light emitting OLED device constructed as set forth in Example 3 (Compound 2:Ir(piq)2acac emissive layer and no wavelength convertor layer) at an intensity of about 20 mW/cm² for about 8.3 minutes.

After the treatment, the number or severity of the lesions is anticipated to be reduced. The treatment is repeated as needed.

### Example 11

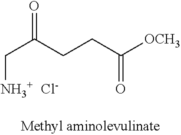

Methyl aminolevulinate (16.8% topical cream, available as METVIXIA® Cream from GALERMA LABORATORIES, Fort Worth, Tex., USA) is topically applied to individual lesions on a person suffering from actinic keratoses. The excess cream is removed with saline, and the lesions are illuminated with the red light emitting OLED constructed as set forth in Example 3 (Compound 2:Ir(piq)2acac) emissive layer and no wavelength convertor layer) emitting at an intensity of about 20 mW/cm² for about 31 minutes for a light dose of about 37 J/cm2. Nitrile gloves are worn at all times during the handling of methyl aminolevulinate. After the treatment, it is anticipated that the number or severity of the lesions is reduced. The treatment is repeated as needed.

### Example 12

Verteporphin is intravenously injected, over a period of about 10 minutes at a rate of about 3 mL/min, to a person suffering from age-related macular degeneration. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light emitting OLED device as set forth in Example 3 (Compound 2:Ir(piq)2acac emissive layer and no wavelength convertor layer) at an intensity of about 20 mW/cm² for about 42 minutes for a total light dose of about 50 J/cm After treatment, the patient's vision is anticipated to be stabilized. The treatment is repeated as needed.

### Example 13

Verteporphin is intravenously injected, over a period of about 10 minute at a rate of about 3 mL/min, to a person suffering from pathological myopia. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light emitting OLED device as set forth in Example 3 (Compound 2:Ir(piq)2acac emissive layer and no wavelength convertor layer) at an intensity of about 20 mW/cm² for about 42 minutes for a total light dose of about 50 J/cm2.

After treatment, the patient's vision is anticipated to be stabilized. The treatment is repeated as needed.

### Example 14

Verteporphin is intravenously injected, over a period of about 10 minutes at a rate of about 3 mL/min, to a person suffering from presumed ocular histoplasmosis. The verteporphin (7.5 mL of 2 mg/mL reconstituted solution, available as Visudyne® from Novartis) is diluted with 5% dextrose to a volume of 30 mL using a sufficient quantity of the reconstituted verteporphin so that the total dose injected is about 6 mg/m² of body surface.

About 15 minutes after the start of the 10 minute infusion of verteporphin, the verteporphin is activated by illuminating the retina with a red light emitting OLED device at an intensity of about 20 mW/cm² for about 42 minutes for a total light dose of about 50 J/cm2. After treatment, the patient's vision is anticipated to be stabilized. The treatment is repeated as needed.

### Example 15

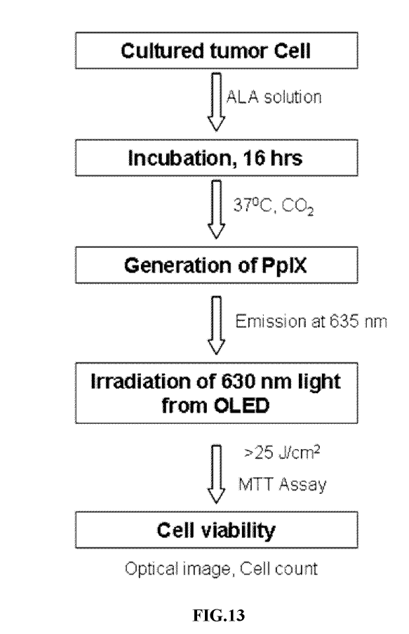

An efficacy study has been performed with 5-aminolevulinic acid (ALA) and CHO-K1 (Chinese Hamster Ovarian Cancer, ATCC, CRL-2243) cell line. FIG. 13 exhibits the efficacy study scheme. Cells were cultured in a 96-well media (Hyclone F-12K medium and dulbeccdo phosphate buffer saline, DPBS) and incubated at 37° C. under CO₂ atmosphere for about 24 hrs. The cells were then calibrated by cell counting with a standard cross area under optical microscope (Olympus IX-70) to establish a base reference number of cells about 10,000 counts in 100 uL medium per well plate. ALA solutions (0.84 mg/mL˜3.3 mg/mL in F-12K medium) with three different concentrations as 0.5 mM, 1 mM, and 2 mM were introduced into same media as mentioned above and incubated for about 16 hrs at 37° C. under CO₂ atmosphere. While not being limited by theory, it is believed that in this process, ALA undergoes a biological transformation and is converted to protoporphyrin IX (PpIX). The generation of PpIX was confirmed by fluorescence emission at 635 nm.

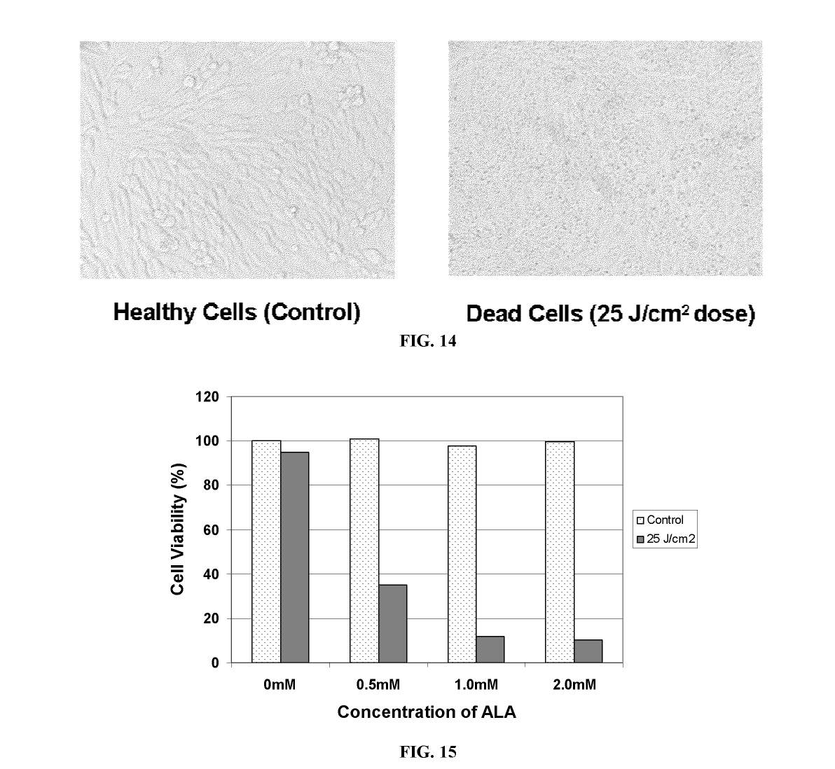

An OLED was constructed similar to that of Example 3 (emissive layer comprising Compound 2:Ir(piq)₂acac was used instead of Compound 2:Ir(ppy)₃). The cells were then irradiated with red light (630 nm) from the OLED with a total dose of 25 J/cm². While not being limited by theory, it is believed PpIX absorbs 630 nm light and is excited to its singlet state followed by intersystem crossing to triplet state. Since the triplet state has longer lifetime, the triplet PpIX interacts with molecular oxygen and generates singlet oxygen and other reactive oxygen species (ROS). These ROS have short lifetime, and thus diffuse only about several tens of nm before reacting with different cell components such as cell membrane, mitocndria, lissome, golgy bodies, nucleus etc. This destroys the cell components, and thus kills the tumor cell. Optical microscope (Olympus IX-70) images of the cells after 25 J/cm² red light irradiation shows (FIG. 14) that the healthy leafy type cells transforms to droplet type upon light irradiation indicating significant cell death.

Followed by light irradiation, 10 uL of MTT solution (Invitrogen, 3,(4,5-dimethylthiazol-2-yl)-2,5-diphenyl tetrazolium bromide, 5 mg/mL in DPBS) was added to each well including the control well and shaken well to mix completely. The wells were incubated (37° C., 5% CO₂) for about 1.5 hrs to generate purple crystals. Then 100 uL MTT solubilization solution were added to each well and incubated (37° C., 5% CO₂) for about 16 hrs to dissolve the purple crystals. Finally the absorbance of the cells at about 570 nm with a reference wavelength at 690 nm were recored by a microplate reader (BioTeK MQX-200) in order to estimate cell viability (%). Cell viability results are shown in FIG. 15. FIG. 15 shows that as the concentration of ALA is increased, the level of cell death also increases. At ALA concentrations of about 1 mM or higher, cell death is about 90% or higher at a near-IR light dose of 25 J/cm². The reference cells were irradiated with same dose of light but without ALA. For a better comparison identical cells were kept at normal environment without light irradiation and compared with reference.

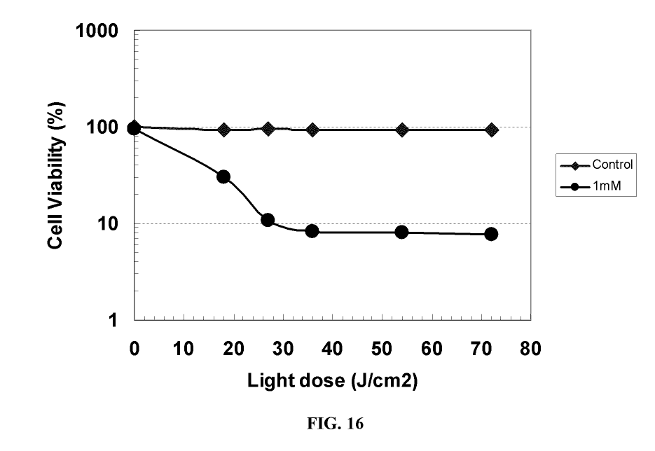

Light Dosimetry was used to optimize the irradiation condition. FIG. 16 shows the cell viability result and compared with the reference. In this case the concentration of ALA was fixed at 1 mM and light dose was varied from 18 J/cm² to 72 J/cm². As shown almost 90% cells were destroyed with a light dose above 25 J/cm², indicating a potential value of OLED for the PDT treatment.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above, but should be determined only by a fair reading of the claims that follow.

What is claimed is:

1. An organic light-emitting device for use in phototherapy comprising:
a light-emitting layer comprising a host compound and an electroluminescent compound;
and
wherein the host compound is represented by Formula 1: 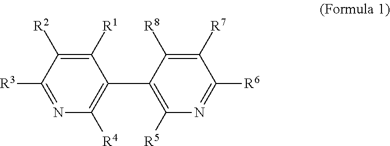
wherein R¹, R², R³, R⁶, R⁷, and R⁸ are independently selected from the group consisting of H, optionally substituted C₁₋₁₂ alkyl, optionally substituted phenyl, optionally substituted carbazolyl, optionally substituted diphenylamine, optionally substituted carbazolylphenyl, and optionally substituted diphenylaminophenyl;
provided that: at least one of R¹, R², and R³ is selected from optionally substituted carbazolyl, optionally substituted diphenylamine, optionally substituted carbazolylphenyl, and optionally substituted diphenylaminophenyl, and at least one of R⁶, R⁷, and R⁸ is selected from optionally substituted carbazolyl, optionally substituted diphenylamine, optionally substituted carbazolylphenyl, and optionally substituted diphenylaminophenyl; and
R⁴ and R⁵ are independently selected from the group consisting of H, optionally substituted C₁₋₁₂ alkyl, optionally substituted phenyl, optionally substituted diphenylamine and optionally substituted diphenylaminophenyl; and
wherein the device is configured to emit a therapeutically effective amount of light to a mammal.

2. The device of claim 1, wherein the electroluminescent compound is selected from tris(2-phenylpyridine)iridium, bis[(1-phenylisoquinolinato-N, C2′)]iridium (III) (acetylacetonate), and 5-trifluoromethyl-2-[3-(N-phenylcarbazolyl)]-pyridine))iridium (III) acetylacetonate, and Bis[4-phenylthieno[3,2-c]pyridine]IrIII acetylacetonate.

3. The device of claim 1, wherein R¹, R³, R⁴, R⁵, R⁶, and R⁸ are H, and R² and R⁷ are independently selected from optionally substituted carbazolyl, optionally substituted diphenylamine, optionally substituted carbazolylphenyl, and optionally substituted diphenylaminophenyl.

4. The device of claim 1, wherein the host compound is further represented by Formula 2: 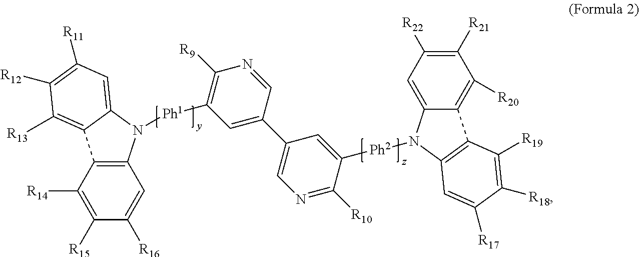
wherein each dotted line is independently an optional bond,
Ph¹ and Ph² are independently optionally substituted 1,4-interphenylene or optionally substituted 1,3-interphenylene,
y and z are independently 0 or 1;
R⁹ and R¹⁰ are independently H, C₁₋₃ alkyl, or C₁₋₃ perfluoroalkyl; and
R¹¹, R¹², R¹³, R¹⁴, R¹⁵, R¹⁶, R¹⁷, R¹⁸, R¹⁹, R²⁰, R²¹, and R²² are independently selected from the group consisting of H, C₁₋₁₂ alkyl, C₁₋₆F₁₋₁₃ fluoroalkyl, and optionally substituted phenyl.

5. The device of claim 4, wherein R¹¹, R¹⁶, R¹⁷, and R²² are independently H or C₁₋₈ alkyl.

6. The device of claim 4, wherein R¹¹, R¹⁶, R¹⁷, and R²² are independently C₁₋₈ alkyl or phenyl.

7. The device of claim 4, wherein the host compound is selected from:

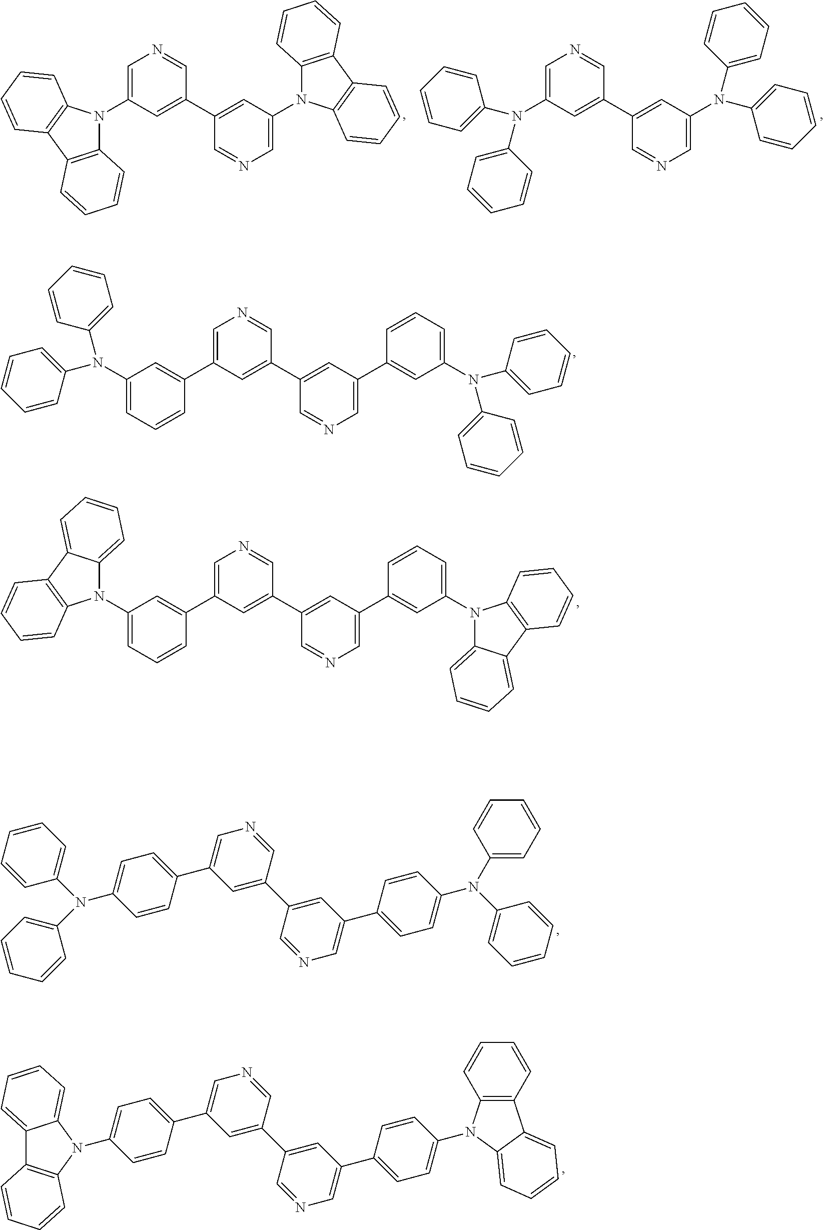

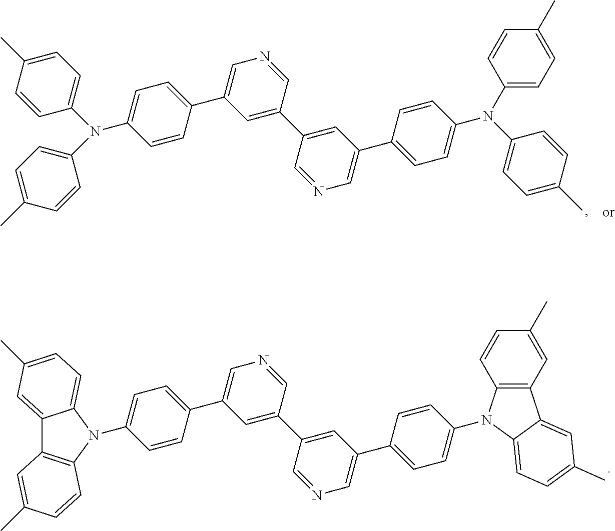

8. The device of claim 4, wherein the host compound is:

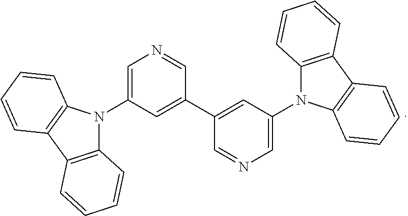

9. A method of carrying out phototherapy comprising:
exposing at least a portion of a tissue of a mammal to light from a device of claim 4.

10. The method of claim 9, wherein the tissue comprises a photosensitive compound which is not naturally in the tissue, and wherein at least a portion of the photosensitive compound is activated by exposing the portion of the tissue to light from the device.

11. A method of treating a disease, comprising:
exposing at least a portion of a tissue of a mammal in need thereof with light from a device of claim 4;
wherein the tissue comprises a photosensitive compound which is not naturally in the tissue, and wherein at least a portion of the photosensitive compound is activated by exposing the portion of the tissue to light from the device to thereby treat the disease.

12. The method of claim 11, wherein activating the photosensitive compound produces singlet oxygen.

13. The method of claim 11, wherein the photosensitive compound is 5-aminolevulinic acid, verteporfin, zinc phthalocyanine, or pharmaceutically acceptable salts thereof.

14. The method of claim 11, wherein the disease is cancer.

15. The method of claim 11, wherein the disease is a microbial infection.

16. The method of claim 11, wherein the disease is a skin condition.

17. The method of claim 11, wherein the disease is an eye condition.

18. An organic light-emitting device for use in phototherapy comprising:
a light-emitting layer comprising an electroluminescent coordination compound comprising a metal-ligand complex, wherein the metal-ligand complex comprises:
a metal selected from platinum and iridium; and
at least 1 ligand selected from the group consisting of optionally substituted acetoacetonate, optionally substituted picolinate, optionally substituted phenylpyridinato, optionally substituted triazolylpyridinato, optionally substituted benzothienylpyridinato, optionally substituted tetrazolylpyridinato, optionally substituted phenylisoquinolinato, optionally substituted tetra(1-pyrazolyl)borate, optionally substituted phenylquinolinyl, optionally substituted phenyloxazolinato, optionally substituted dibenzoquinoxalino, optionally substituted thiophenylisoquinolinato, optionally substituted 2,5-bis-(2′-fluorene)pyridine, optionally substituted phenylbenzothiazolato, optionally substituted fluorenylisoquinolinato, optionally substituted thienylpyridinato, optionally substituted phenylcarbazolylpyridinato, and optionally substituted carbazolylphenylpyridinato;
a wavelength convertor comprising: yttrium aluminum garnet, yttria, titania or alumina, and at least one dopant which is an atom or an ion of an element selected from the group consisting of Cr, Ce, Gd, La, Tb, Pr, Sm, and Eu; and
wherein the wavelength convertor is configured to receive at least a portion of light emitted from the organic light-emitting diode in a wavelength range of about 350 nm to about 600 nm and convert at least a portion of the light received to light in a wavelength range of about 600 nm to about 800 nm.

19. The device of claim 18, wherein all ligands in the electroluminescent coordination compound are selected from the group consisting of optionally substituted acetoacetonate, optionally substituted picolinate, optionally substituted phenylpyridinato, optionally substituted triazolylpyridinato, optionally substituted benzothienylpyridinato, optionally substituted tetrazolylpyridinato, optionally substituted phenylisoquinolinato, optionally substituted tetra(1-pyrazolyl)borate, optionally substituted phenylquinolinyl, optionally substituted phenyloxazolinato, optionally substituted dibenzoquinoxalino, optionally substituted thiophenylisoquinolinato, optionally substituted 2,5-bis-(2′-fluorene)pyridine, optionally substituted phenylbenzothiazolato, optionally substituted fluorenylisoquinolinato, optionally substituted thienylpyridinato, optionally substituted phenylcarbazolylpyridinato, optionally substituted phenylthienylpyridinato and optionally substituted carbazolylphenylpyridinato.

20. The device of claim 18, wherein the wavelength convertor comprises Ce doped yttrium aluminum garnet.
